SEQUENCE LISTING (30 sequences)

SEQ ID NO: 1 - DNA, 24 bp, synthetic construct
  misc_feature 1..24: note = Description of Artificial Sequence: Synthetic oligonucleotide
  source 1..24: mol_type = other DNA
ccggggaagctgtccgggctccga

SEQ ID NO: 2 - DNA, 24 bp, synthetic construct
  misc_feature 1..24: note = Description of Artificial Sequence: Synthetic oligonucleotide
  source 1..24: mol_type = other DNA
aaactcggagcccggacagcttcc

SEQ ID NO: 3 - DNA, 24 bp, synthetic construct
  misc_feature 1..24: note = Description of Artificial Sequence: Synthetic oligonucleotide
  source 1..24: mol_type = other DNA
ccggcatcgagtgtcgaatatgtg

SEQ ID NO: 4 - DNA, 24 bp, synthetic construct
  misc_feature 1..24: note = Description of Artificial Sequence: Synthetic oligonucleotide
  source 1..24: mol_type = other DNA
aaaccacatattcgacactcgatg

SEQ ID NO: 5 - DNA, 20 bp, synthetic construct
  misc_feature 1..20: note = Description of Artificial Sequence: Synthetic primer
  source 1..20: mol_type = other DNA
gcagcttggcaccttctgtg

SEQ ID NO: 6 - DNA, 20 bp, synthetic construct
  misc_feature 1..20: note = Description of Artificial Sequence: Synthetic primer
  source 1..20: mol_type = other DNA
gatgacagagccctcggagc

SEQ ID NO: 7 - DNA, 24 bp, synthetic construct
  misc_feature 1..24: note = Description of Artificial Sequence: Synthetic primer
  source 1..24: mol_type = other DNA
gagtgtcgaatatgtggggacaag

SEQ ID NO: 8 - DNA, 20 bp, synthetic construct
  misc_feature 1..20: note = Description of Artificial Sequence: Synthetic primer
  source 1..20: mol_type = other DNA
gcaacctgccctagactgtc

SEQ ID NO: 9 - DNA, 20 bp, Mus musculus
  source 1..20: mol_type = genomic DNA
cagctttgcctcactcagga

SEQ ID NO: 10 - DNA, 20 bp, Mus musculus
  source 1..20: mol_type = genomic DNA
gaggcaggtgtttctctccc

SEQ ID NO: 11 - DNA, 21 bp, Mus musculus
  source 1..21: mol_type = genomic DNA
ggcctctgtggtacacgacaa

SEQ ID NO: 12 - DNA, 21 bp, Mus musculus
  source 1..21: mol_type = genomic DNA
ctcagtgggagcgactcttca

SEQ ID NO: 13 - DNA, 20 bp, Mus musculus
  source 1..20: mol_type = genomic DNA
agctggtggtggaatgtgtt

SEQ ID NO: 14 - DNA, 20 bp, Mus musculus
  source 1..20: mol_type = genomic DNA
ttcctttggctcatgccctt

SEQ ID NO: 15 - DNA, 24 bp, Mus musculus
  source 1..24: mol_type = genomic DNA
tcttggctacagcaaggccagata

SEQ ID NO: 16 - DNA, 24 bp, Mus musculus
  source 1..24: mol_type = genomic DNA
agctatgcagcatggaacatgacg

SEQ ID NO: 17 - DNA, 19 bp, Mus musculus
  source 1..19: mol_type = genomic DNA
gacgcctgaacggctctgt

SEQ ID NO: 18 - DNA, 21 bp, Mus musculus
  source 1..21: mol_type = genomic DNA
tctccgaagccatccttgtag

SEQ ID NO: 19 - DNA, 20 bp, Mus musculus
  source 1..20: mol_type = genomic DNA
ccttccgtcgtcttctgtgt

SEQ ID NO: 20 - DNA, 20 bp, Mus musculus
  source 1..20: mol_type = genomic DNA
ccatcaaacctgttgagcgg

SEQ ID NO: 21 - DNA, 20 bp, Mus musculus
  source 1..20: mol_type = genomic DNA
ggtgttcgcagctgttttgg

SEQ ID NO: 22 - DNA, 20 bp, Mus musculus
  source 1..20: mol_type = genomic DNA
ggtgagatacgcccaaatgc

SEQ ID NO: 23 - DNA, 20 bp, Mus musculus
  source 1..20: mol_type = genomic DNA
cactctcgctttctggaggg

SEQ ID NO: 24 - DNA, 20 bp, Mus musculus
  source 1..20: mol_type = genomic DNA
acgcgcttgtacccattgat

SEQ ID NO: 25 - DNA, 20 bp, synthetic construct
  misc_feature 1..20: note = Description of Artificial Sequence: Synthetic primer
  source 1..20: mol_type = other DNA
gatctcatggagcccagact

SEQ ID NO: 26 - DNA, 20 bp, synthetic construct
  misc_feature 1..20: note = Description of Artificial Sequence: Synthetic primer
  source 1..20: mol_type = other DNA
ttgtgctgattcatgcctgt

SEQ ID NO: 27 - DNA, 20 bp, synthetic construct
  misc_feature 1..20: note = Description of Artificial Sequence: Synthetic primer
  source 1..20: mol_type = other DNA
ttcactgggtgctcgggaag

SEQ ID NO: 28 - DNA, 21 bp, synthetic construct
  misc_feature 1..21: note = Description of Artificial Sequence: Synthetic primer
  source 1..21: mol_type = other DNA
tggcattgtcgcaaggataac

SEQ ID NO: 29 - DNA, 20 bp, synthetic construct
  misc_feature 1..20: note = Description of Artificial Sequence: Synthetic primer
  source 1..20: mol_type = other DNA
gcaaccctctcccatcagtg

SEQ ID NO: 30 - DNA, 20 bp, synthetic construct
  misc_feature 1..20: note = Description of Artificial Sequence: Synthetic primer
  source 1..20: mol_type = other DNA
gcctaacaccgtgcttctca

(19) United States
(12) Patent Application Publication
(10) Pub. No.: US 2025/0281460 A1
(43) Pub. Date: Sep. 11, 2025

(54) PEGYLATED BILIRUBIN FOR THE TREATMENT OF HYPERLIPIDEMIA, OBESITY, FATTY LIVER DISEASE, CARDIOVASCULAR DISEASES AND TYPE II DIABETES

(71) Applicants: The University of Toledo, Toledo, OH (US); University of Mississippi Medical Center, Jackson, MS (US)

(72) Inventors: Terry D. Hinds, JR., Toledo, OH (US); David E. Stec, Jackson, MS (US)

(73) Assignees: The University of Toledo, Toledo, OH (US); University of Mississippi Medical Center, Jackson, MS (US)

(21) Appl. No.: 19/092,515

(22) Filed: Mar. 27, 2025

Related U.S. Application Data
(62) Division of application No. 17/433,772, filed on Aug. 25, 2021.
(60) Provisional application No. 62/809,906, filed on Feb. 25, 2019.
(60) Provisional application No. 62/891,046, filed on Aug. 23, 2019.

(51) Int. Cl. A61K 31/409; A61K 9/51; A61P 1/16; A61P 3/04; A61P 3/06; A61P 3/10

(52) U.S. Cl. CPC A61K 31/409; A61K 9/51; A61P 1/16; A61P 3/04; A61P 3/06; A61P 3/10

(57) ABSTRACT

Compositions and methods for the treatment of obesity, hyperlipidemia, fatty liver disease, cardiovascular disease and type II diabetes are described. Also described are compositions and methods for decreasing one or more of body weight, total fat, percent fat mass, visceral fat, epididymal fat, hepatic fat content, fasting blood glucose, decreasing white adipose fat (WAT) adipocyte size, or increasing percent lean mass. The compositions and methods involve PEGylated bilirubin.

### RELATED APPLICATIONS

This is a divisional application which claims priority to U.S. application Ser. No. 17/433,772, filed under 35 U.S.C. § 371 on Aug. 25, 2021, published; which is the national phase entry of international application PCT/US2020/018604, filed under the authority of the Patent Cooperation Treaty on Feb. 18, 2020, published; which claims priority to U.S. Provisional Application No. 62/809,906 filed under 35 U.S.C. § 111 (b) on Feb. 25, 2019, and U.S. Provisional Application No. 62/891,046 filed under 35 U.S.C. § 111 (b) on Aug. 23, 2019. The entire disclosure of each of the aforementioned applications is incorporated herein by references for all purposes.

### STATEMENT REGARDING FEDERALLY SPONSORED RESEARCH

This invention was made with no government support. The government has no rights in this invention.

### SEQUENCE LISTING

The instant application contains a Sequence Listing which has been submitted via Patent Center and is hereby incorporated by reference in its entirety. The XML copy, created on Mar. 21, 2025, is named 420_70854_SEQ_LIST_D2018-40-2.xml, and is 33,135 bytes in size.

### BACKGROUND

Type II diabetes result in significant health spending. However, to date, no drug has demonstrated sustainable efficacy in the treatment of type II diabetes. Thus, there is a need in the art for new methods and compositions useful for the treatment of type II diabetes.

### SUMMARY

Provided is a method for decreasing one or more of body weight, total fat, percent fat mass, visceral fat, epididymal fat, hepatic fat content, fasting blood glucose, low density lipoprotein (LDL) cholesterol, very low density lipoprotein (VLDL), ApoB-VLDL, and plasma triglyceride levels, the method comprising administering an effective amount of PEGylated bilirubin to a subject, and decreasing one or more of body weight, total fat, percent fat mass, visceral fat, epididymal fat, hepatic fat content, fasting blood glucose, LDL cholesterol, plasma triglyceride levels, VLDL, and ApoB-VLDL in the subject. In certain embodiments, the subject is a human. In certain embodiments, the PEGylated bilirubin comprises bilirubin nanoparticles.

Further provided is a method for increasing percent lean mass, the method comprising administering an effective amount of PEGylated bilirubin to a subject, and increasing percent lean mass in the subject. In certain embodiments, the subject is a human. In certain embodiments, the PEGylated bilirubin comprises bilirubin nanoparticles.

Further provided is a method for decreasing white adipose fat (WAT) adipocyte size, the method comprising administering an effective amount of PEGylated bilirubin to a subject, and decreasing WAT adipocyte size of the WAT cells in the subject. In certain embodiments, the subject is a human. In certain embodiments, the PEGylated bilirubin comprises bilirubin nanoparticles.

Further provided is a method for decreasing hepatic fat content, the method comprising administering an effective amount of PEGylated bilirubin to a subject, and decreasing lipid content in the liver of the subject. In certain embodiments, the subject is a human. In certain embodiments, the PEGylated bilirubin comprises bilirubin nanoparticles.

Further provided is a method for increasing expression of UCP1 or ADRB3 in WAT, the method comprising administering an effective amount of PEGylated bilirubin to WAT cells, and increasing expression of UCP1 or ADRB3 in the WAT cells. In certain embodiments, the PEGylated bilirubin comprises bilirubin nanoparticles. In certain embodiments, the subject is a human.

Further provided is a method for increasing mitochondrial function and number in WAT cells, the method comprising administering an effective amount of PEGylated bilirubin to WAT cells and increasing mitochondrial function and number in the WAT cells. In certain embodimients, the PEGylated bilirubin comprises bilirubin nanoparticles.

Further provided is a method of treating type II diabetes, hyperlipidemia, obesity, or cardiovascular disease in a subject, the method comprising administering an effective amount of PEGylated bilirubin to a subject having type II diabetes, hyperlipidemia, obesity, or cardiovascular disease, and treating the type II, hyperlipidemia, obesity, or cardiovascular disease in the subject. In certain embodiments, the PEGylated bilirubin comprises bilirubin nanoparticles. In certain embodiments, the subject is a human.

Further provided is a method of reducing one or more of plasma triglycerides, very low density lipoprotein (VLDL), ApoB-VLDL, or low density lipoprotein (LDL) cholesterol in a subject, the method comprising administering an effective amount of PEGylated bilirubin to a subject and reducing one or more of plasma and liver triglycerides, very low density lipoprotein (VLDL), ApoB-VLDL, or low density lipoprotein (LDL) cholesterol in the subject. In certain embodiments, the PEGylated bilirubin comprises bilirubin nanoparticles. In certain embodiments, the subject is a human.

Further provided is a method of increasing ApoA1 or high density lipoprotein (HDL) cholesterol in a subject, the method comprising administering an effective amount of PEGylated bilirubin to a subject and increasing ApoA1 or HDL cholesterol in the subject. In certain embodiments, the PEGylated bilirubin comprises bilirubin nanoparticles. In certain embodiments, the subject is a human.

Further provided is a composition comprising polyethylene glycol covalently attached to bilirubin for use in the production of a medicament for decreasing one or more of body weight, total fat, percent fat mass, visceral fat, epididymal fat, hepatic fat content, fasting blood glucose, VLDL, ApoB-VLDL, and LDL cholesterol, or increasing mitochondrial function and number in WAT cells, or increasing ApoA1 or HDL cholesterol, or treating or preventing type II diabetes, fatty liver disease, hyperlipidemia, obesity, or cardiovascular disease. In certain embodiments, the composition comprises bilirubin nanoparticles.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A: Biliverdin (precursor to bilirubin) treatments significantly reduced lipid accumulation at 10 μM and 50 μM.

FIG. 1B: Biliverdin at 50 M substantially decreased lipid accumulation, and significantly increased mitochondrial and lipid burning genes Ucp1 and Cpt1 mRNA expression.

FIG. 1C: Biliverdin and WY 14,643 significantly increased the mitochondrial oxygen consumption rate (OCR) for maximum respiration.

FIG. 1D: Biliverdin significantly increased PPARα occupancy at the 13K enhancer of the Ucp1 and the −3306 to −3109 region of the Cpt1 promoter.

FIG. 2A: Biliverdin treatments in 3T3-PPARα cells that overexpressed PPARα caused significantly higher maximum respiration, basal respiration, proton leak, and ATP production compared to control.

FIG. 2B: 3T3-PPARγ2 did not have significant increase in OCR or gene related activity (FIG. 2C).

FIG. 2C: 3T3-PPARγ2 did not have significant increase in gene related activity.

FIG. 3A: Energy expenditure was evaluated by SeaHorse analysis in a murine BAT cell line treated with biliverdin, rosiglitazone, WY 14,643.

FIG. 3B: Increasing doses of biliverdin over the differentiation of the BAT cells had no impact on lipid accumulation despite increasing mitochondrial function.

FIG. 3C: Treatment with 50 μM biliverdin, 50 μM WY14,463, or 10 μM rosiglitazone in differentiated BAT cells for 24 hrs caused a significant increase in Ucp1 and Adrb3 mRNA with all three ligands.

FIGS. 3D-3E: The proximal promoter had no response with or without PPARα expressed in Cos7 cells.

FIGS. 4A-4C: BAT PPARα CRISPR KO cells (clone 1 and 2) and wild-type (WT) cells were treated with biliverdin or WY 14,643 for 24 hours and the impact on mitochondrial function was determined via Seahorse analysis. The WT BAT cells responded with increased OCR with WY 14,643 and biliverdin for maximum respiration, basal respiration, and ATP production.

FIGS. 5A-5F: Bilirubin, fenofibrate, and WY 14,643 mitigate binding of the human PPARα LBD to coregulator motifs (FIG. 5A). FIG. 5B shows the molecular signatures of bilirubin and fenofibrate were also similar. The highest 40 and lowest 25 coregulator binding affinities subtracted from the vehicle were sorted to remove the background (FIGS. 5C-5F). FIG. 5E shows Venn diagrams for the highest and lowest interactions of bilirubin, WY 14,643, and fenofibrate.

FIGS. 6A-6H: PEG-BR treated mice have reduced adipocyte size in WAT and higher mitochondria function. FIG. 6A shows total bilirubin levels in mice control vs 4wk treated PEG-BR treated. FIG. 6B shows blood glucose. FIG. 6C shows body weight, total fat, % fat mass, % visceral fat, % ependymal fat, and % lean mass in control mice (gray) vs PEG-BR (yellow). FIG. 6D shows white adipose tissue (WAT) adipocyte size. FIG. 6E shows brown adipose tissue (BAT) adipocyte size. FIGS. 6D-6E further show mitochondria function measured via Mitotracker (green) in WAT tissues of control vs PEG-BR mice, and densitometry, in WAT (FIG. 6D) and BAT (FIG. 6E). FIGS. 6F-6G show UCP1 mRNA, ADRB3 mRNA, and PPARα mRNA expression in WAT (FIG. 6F) and BAT (FIG. 6G). *, P<0.05 or **, P<0.01, ***, P<0.001 vs Veh. FIG. 6H shows the highest 40 and lowest 25 coregulator binding affinities subtracted from the vehicle to remove the background.

FIGS. 7A-7B: PEG-bilirubin decreases plasma triglycerides, very low density lipoprotein (VLDL), ApoB-VLDL, and low density lipoprotein (LDL) cholesterol, and increases ApoA1, and high density lipoprotein (HDL) cholesterol.

FIGS. 7A-7C: Graphs showing effects on metabolic parameters, lipoproteins composition, triglyceride distribution, VLDL triglyceride subfractions, LDL triglyceride subfractions, HDL triglycerides distribution, cholesterol distribution, VLDL cholesterol subfractions, LDL cholesterol subfractions, HDL cholesterol distribution, free cholesterol distribution, VLDL free cholesterol subfractions, LDL free cholesterol subfractions, HDL free cholesterol distribution, phospholipid distribution, VLDL phospholipid subfractions, LDL phospholipid subfractions, and HDL phospholipid distribution. FIG. 7A shows PEG-bilirubin decreases plasma triglycerides, very low density lipoprotein (VLDL), ApoB-VLDL, and low density lipoprotein (LDL) cholesterol, and increases ApoA1, and high density lipoprotein (HDL) cholesterol.

FIG. 8: Graphs showing effects on metabolic parameters, lipoproteins composition, ApoA1 distribution, and ApoA1 distribution.

FIGS. 9A-9C 9A-9D: Mice with hyperbilirubinemia have increased phosphorylation of Ser21 PPARα and PPARα target genes in adipose. FIG. 9A shows WAT adipocyte size and mitochondria number in the Gilbert's and control mice. Genes were measured by Real-time PCR from WAT (FIG. 9B) and BAT (FIG. 9C) of the UGT*28 mice. WT, n=5, Gilbert's, n=4.

PRIOR ART FIG. 10: Non-limiting example synthesis of PEGylated bilirubin.

FIGS. 11A-11B: ¹H NMR spectra of PEG-BR.

FIG. 12: IR spectrum of PEG-BR.

FIG. 13: Mass spectrum of PEG-BR.

FIGS. 14A-14B: PEG-BR Treatment decreases hepatic lipid accumulation: FIG. 14A shows percent hepatic fat; FIG. 14B shows hepatic triglycerides (mg/g).

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

Understanding of bilirubin has been shaped by the dramatic consequences of extreme hyperbilirubinemia seen in pathological jaundice and Crigler-Najjar syndrome. This led to the idea that bilirubin is categorically harmful. However, there is compelling evidence that hypobilirubinemia (lower end and below normal levels) are also deleterious and lead to metabolic deficits. Several large population studies have reflected a negative correlation between serum bilirubin levels with body weight and plasma glucose levels. People exhibiting mildly elevated (>12 μM) bilirubin levels have significantly fewer metabolic disorders such as obesity or type II diabetes. Thus, there may be significant differences reflected in various adipose stores or molecular signaling pathways.

In humans and rodents, adipose tissue depots have different functions, especially in the adipokine hormones that are secreted. White adipose tissue (WAT) is located mostly in the visceral portion (i.e., near visceral organs) and subcutaneous (thighs and stomach), and expands during obesity and secretes adipokines that release inflammatory factors. WAT is high in lipid storage. Brown adipose tissue (BAT) is located in the back of the neck, mediastrinum, and adrenal glands. BAT is high in lipid burning capacity. BAT produces hormones that reduce inflammation and increase energy expenditure. The nuclear receptor peroxisome proliferator-activated receptor a (PPARa) has been shown to be important for the development of BAT and a ‘browning’ of WAT. Pharmacological stimuli can increase PPARα in WAT causing browning which reduces body weight.

Bilirubin increases the transcriptional activity of PPARα at a minimal promoter and endogenous genes. Compounds that target the PPARs may simultaneously activate all three PPARs (PPAR pan agonists) or can have selective modulation of a single PPAR (SPPARM). The latter may be a potent inducer of some activities with reduced unwanted effects. Without wishing to be bound by theory, it is believed that there is a relationship between bilirubin and PPARa, and that bilirubin may be a ligand for PPARs. There is very little known on how bilirubin affects WAT or other peripheral tissues, or directs signaling mechanisms.

In the examples herein, bilirubin was evaluated for whether it may serve as a metabolic hormone since it flows through blood and may have a direct action on a target (PPARs) to lessen fat storage and increase adipocyte function. The effects of the lipid-burning capacity of bilirubin on WAT or BAT is unknown. It would be advantageous to comprehensively map the hormonal responses of bilirubin in adipose tissues and determine if its actions are selective on the PPAR isoforms. Activation of the browning of WAT by increasing energy expenditure and the burning of fat has significant implications in reducing adiposity and insulin resistance. Mostly, these processes are mediated by mitochondrial uncoupling proteins during physical activity or brown fat-mediated thermogenesis. During thermogenesis, β3 adrenergic receptor (ADRB3) signaling activates the uncoupling protein 1 (UCP1) to cause protons to leak across the inner mitochondrial membrane increasing oxygen consumption, which overall increases mitochondrial function and fat utilization reversing adipocyte dysfunction. Even though bilirubin reduces body weight, its role in mitochondrial function is unknown. It is shown herein that bilirubin has direct binding to PPARa, and this causes recruitment of a specific set of coregulators which induces mitochondrial function decreasing WAT size, ultimately affecting organismal metabolic balance and glucose homeostasis. Taken together, these findings indicate that bilirubin is a metabolic hormone that controls WAT tissue expansion to lessen hypertrophy and glucose intolerance. Further, bilirubin reduces cholesterol and triglycerides.

Bilirubin has the following structural formula (I):

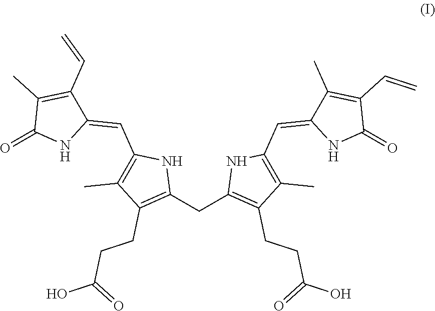 [SMILES: C=CC1=C(C)/C(=C/c2[nH]c(Cc3[nH]c(/C=C4\NC(=O)C(C)=C4C=C)c(C)c3CCC(=O)O)c(CCC(=O)O)c2C)NC1=O]

In comparison, the known PPARα ligands WY-14,643 and fenofibrate have the following structural formulas (II) and (III), respectively:

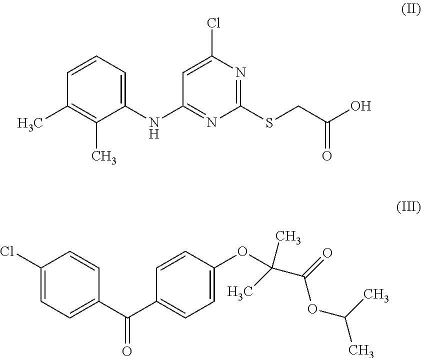 [SMILES: Cc1cccc(Nc2cc(Cl)nc(SCC(=O)O)n2)c1C ; CC(C)OC(=O)C(C)(C)Oc1ccc(C(=O)c2ccc(Cl)cc2)cc1]

Bilirubin activates PPARa, and binds directly to PPARα to reduce lipid accumulation. Bilirubin also increases UCP1 and ADRB3. Epidemiological studies have shown that patients with higher plasma bilirubin exhibit lower body weights, diabetes, and cardiovascular disease. However, thereapeutic uses of bilirubin are problematic because of bilirubin's insolubility in water.

In accordance with the present disclosure, a solubility-enhancing compound being covalently attached to bilirubin may produce a water-soluble compound useful for the same therapeutic purposes of bilirubin. For example, polyethylene glycol (PEG) may be covalently attached to bilirubin, yielding PEGylated bilirubin (PEG-BR). A non-limiting example synthesis of PEG-BR is depicted in PRIOR ART FIG. 10. Bilirubin nanoparticles may form by self-assembly of PEG-BR. As used herein, the term “PEGylated bilirubin” or “PEG-BR” encompasses bilirubin nanoparticles formed from PEG-BR, but does not necessarily require bilirubin nanoparticles. Rather, PEGylated bilirubin may include any compound or composition having a polyethylene glycol covalently attached to bilirubin.

As will be appreciated by those skilled in the art, PEG may come in many forms. PEG generally has the formula of H—(O—CH₂—CH₂)ₙ—OH, where n ranges from 2 to 20,000. PEG compounds may be prepared, for instance, by the polymerization of ethylene oxide. PEG compounds may also be available with different geometries. Furthermore, the PEG compound may be substituted or unsubstitued. The identity of the PEG compound used to form PEGylated bilirubin is not particularly limited.

In one non-limiting example, PEGylated bilirubin has the following structural formula (IV):

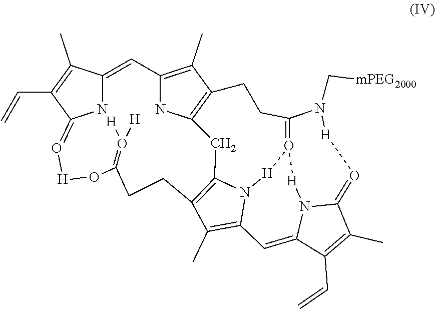

As shown in the examples herein, PEGylated bilirubin reduces blood glucose and body weight in obese mice. PEGylated bilirubin treatment in obese mice increases UCP1 and ADRB3 in WAT. PEGylated bilirubin also reduces plasma triglycerides, very low density lipoprotein (VLDL), ApoB-VLDL, and low density lipoprotein (LDL) cholesterol. PEGylated bilirubin also increases ApoA1, high density lipoprotein (HDL) cholesterol. In accordance with the present disclosure, PEGylated bilirubin may be useful for decreasing body weight, % fat mass, total fat, visceral fat, epididymal fat, and fasting blood glucose, and increasing % lean mass. PEGylated bilirubin may also be useful for decreasing WAT adipocyte size without changing BAT adipocyte size. PEGylated bilirubin may also be useful for reducing blood glucose, body weight, plasma triglycerides, VLDL, ApoB-VLDL, or LDL cholesterol, increasing UCP1 and ADRB3 in WAT, and increasing ApoA1, and HDL cholesterol. In sum, PEGylated bilirubin has lipid burning and glucose lowering properties, and also white adipose tissue remodeling properties to make WAT more brown fat-like and thereby increasing energy expenditure. PEGylated bilirubin may be useful for the treatment of dyslipidemia, obesity, fatty liver disease, and type II diabetes. Furthermore, PEGylated bilirubin may be useful for the treatment of cardiovascular disease because PEGylated bilirubin reduces LDL cholesterol and triglycerides and increases heart-healthy ApoA1 and HDL cholesterol.

Pharmaceutical compositions of the present disclosure comprise an effective amount of a PEGylated bilirubin (an “active” compound), and/or additional agents, dissolved or dispersed in a pharmaceutically acceptable carrier. The preparation of a pharmaceutical composition that contains at least one compound or additional active ingredient will be known to those of skill in the art in light of the present disclosure, as exemplified by Remington's Pharmaceutical Sciences, 2003, incorporated herein by reference. Moreover, for animal (e.g., human) administration, it is understood that preparations should meet sterility, pyrogenicity, general safety, and purity standards as required by FDA Office of Biological Standards.

A composition disclosed herein may comprise different types of carriers depending on whether it is to be administered in solid, liquid or aerosol form, and whether it need to be sterile for such routes of administration as injection. Compositions disclosed herein can be administered intravenously, intradermally, transdermally, intrathecally, intraarterially, intraperitoneally, intranasally, intravaginally, intrarectally, intraosseously, periprosthetically, topically, intramuscularly, subcutaneously, mucosally, intraosseosly, periprosthetically, in utero, orally, topically, locally, via inhalation (e.g., aerosol inhalation), by injection, by infusion, by continuous infusion, by localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 2003, incorporated herein by reference).

The actual dosage amount of a composition disclosed herein administered to an animal or human patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a composition herein and/or additional agent is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsules, they may be compressed into tablets, or they may be incorporated directly with the food of the diet.

In further embodiments, a composition described herein may be administered via a parenteral route. As used herein, the term “parenteral” includes routes that bypass the alimentary tract. Specifically, the pharmaceutical compositions disclosed herein may be administered, for example but not limited to, intravenously, intradermally, intramuscularly, intraarterially, intrathecally, subcutaneous, or intraperitoneally (U.S. Pat. Nos. 6,753,514, 6,613,308, 5,466,468, 5,543,158; 5,641,515, and 5,399,363 are each specifically incorporated herein by reference in their entirety).

Solutions of the compositions disclosed herein as free bases or pharmacologically acceptable salts may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In some cases, the form must be sterile and must be fluid to the extent that easy injectability exists. It should be stable under the conditions of manufacture and storage and should be preserved against the contaminating action of microorganisms, such as bacteria and fungi. The carrier can be a solvent or dispersion medium containing, for example, water, ethanol, polyol (i.e., glycerol, propylene glycol, liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and antifungal agents, such as, but not limited to, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption such as, for example, aluminum monostearate or gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Sterile injectable solutions are prepared by incorporating the compositions in the required amount in the appropriate solvent with various other ingredients enumerated above, as required, followed by filtered sterilization. Generally, dispersions are prepared by incorporating the various sterilized compositions into a sterile vehicle which contains the basic dispersion medium and the required other ingredients from those enumerated above. In the case of sterile powders for the preparation of sterile injectable solutions, some methods of preparation are vacuum-drying and freeze-drying techniques which yield a powder of the active ingredient plus any additional desired ingredient from a previously sterile-filtered solution thereof. A powdered composition is combined with a liquid carrier such as, but not limited to, water or a saline solution, with or without a stabilizing agent.

In other embodiments, the compositions may be formulated for administration via various miscellaneous routes, for example, topical (i.e., transdermal) administration, mucosal administration (intranasal, vaginal, etc.) and/or via inhalation.

Pharmaceutical compositions for topical administration may include the compositions formulated for a medicated application such as an ointment, paste, cream, or powder. Ointments include all oleaginous, adsorption, emulsion, and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones, and luarocapram. Possible bases for compositions for topical application include polyethylene glycol, lanolin, cold cream, and petrolatum, as well as any other suitable absorption, emulsion, or water-soluble ointment base. Topical preparations may also include emulsifiers, gelling agents, and antimicrobial preservatives as necessary to preserve the composition and provide for a homogenous mixture. Transdermal administration of the compositions may also comprise the use of a “patch.” For example, the patch may supply one or more compositions at a predetermined rate and in a continuous manner over a fixed period of time.

In certain embodiments, the compositions may be delivered by eye drops, intranasal sprays, inhalation, and/or other aerosol delivery vehicles. Methods for delivering compositions directly to the lungs via nasal aerosol sprays has been described in U.S. Pat. Nos. 5,756,353 and 5,804,212 (each specifically incorporated herein by reference in their entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts and could be employed to deliver the compositions described herein. Likewise, transmucosal drug delivery in the form of a polytetrafluoroetheylene support matrix is described in U.S. Pat. No. 5,780,045 (specifically incorporated herein by reference in its entirety), and could be employed to deliver the compositions described herein.

It is further envisioned the compositions disclosed herein may be delivered via an aerosol. The term aerosol refers to a colloidal system of finely divided solid or liquid particles dispersed in a liquefied or pressurized gas propellant. The typical aerosol for inhalation consists of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight, and the severity and response of the symptoms.

In particular embodiments, the compounds and compositions described herein are useful for treating, preventing, or ameliorating obesity, hyperlipidemia, cardiovascular disease, and type II diabetes, for decreasing one or more of body weight, total fat, percent fat mass, visceral fat, epididymal fat, fasting blood glucose, white adipose fat (WAT) adipocyte size, plasma triglycerides, VLDL, ApoB-VLDL, or LDL cholesterol, or for increasing expression of UCP1 or ADRB3 in white adipose fat (WAT), or increasing ApoA1, or HDL cholesterol. Furthermore, the compounds and compositions herein can be used in combination therapies. That is, the compounds and compositions can be administered concurrently with, prior to, or subsequent to one or more other desired therapeutic or medical procedures or drugs. The particular combination of therapies and procedures in the combination regimen will take into account compatibility of the therapies and/or procedures and the desired therapeutic effect to be achieved. Combination therapies include sequential, simultaneous, and separate administration of the active compound in a way that the therapeutic effects of the first administered procedure or drug is not entirely disappeared when the subsequent procedure or drug is administered.

It is further envisioned that the compounds and methods described herein can be embodied in the form of a kit or kits. A non-limiting example of such a kit is a kit for making a PEGylated bilirubin, the kit comprising bilirubin and polyethylene glycol in separate containers, where the containers may or may not be present in a combined configuration. Many other kits are possible, such as kits further comprising a cosolvent, or further comprising a pharmaceutically acceptable carrier, diluent, or excipient. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

### Results

### Bilirubin Reduces Lipids in White Adipocytes by Increasing Mitochondrial Function

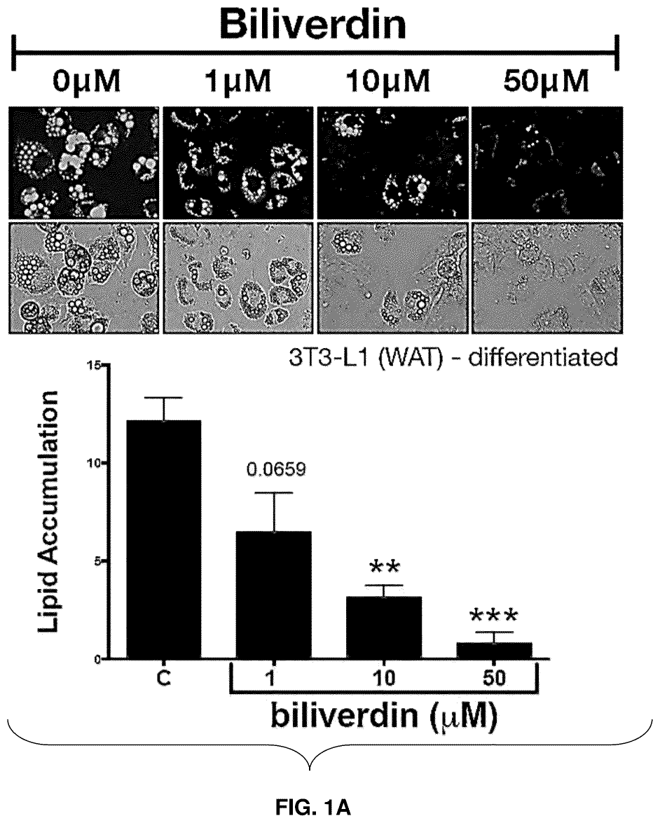

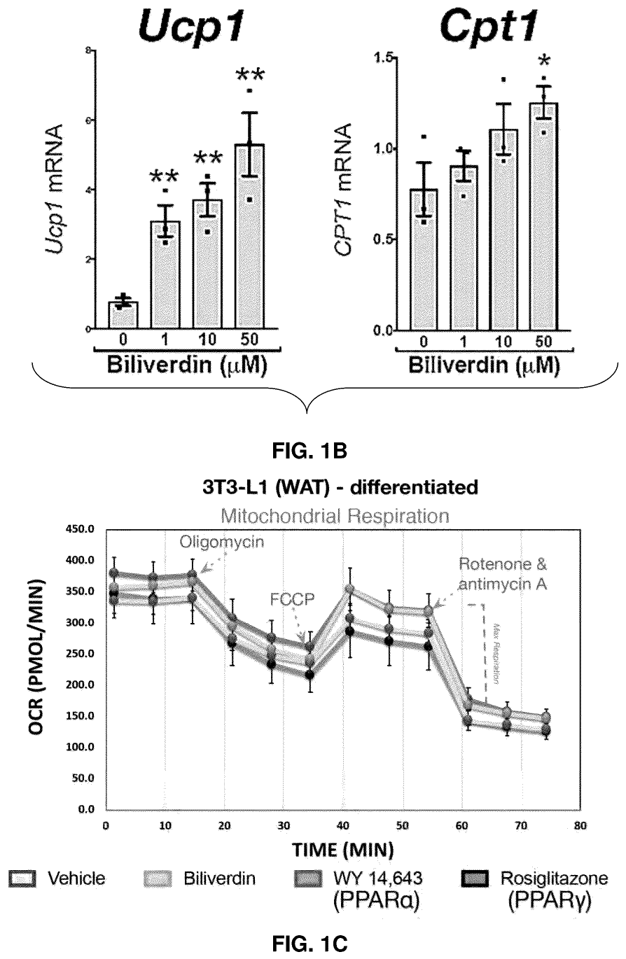

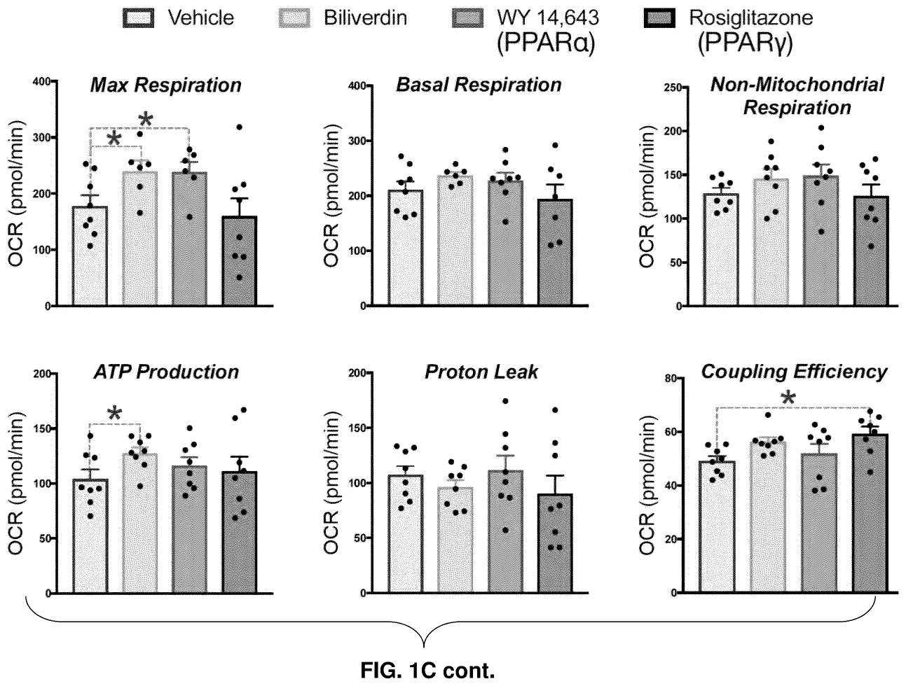

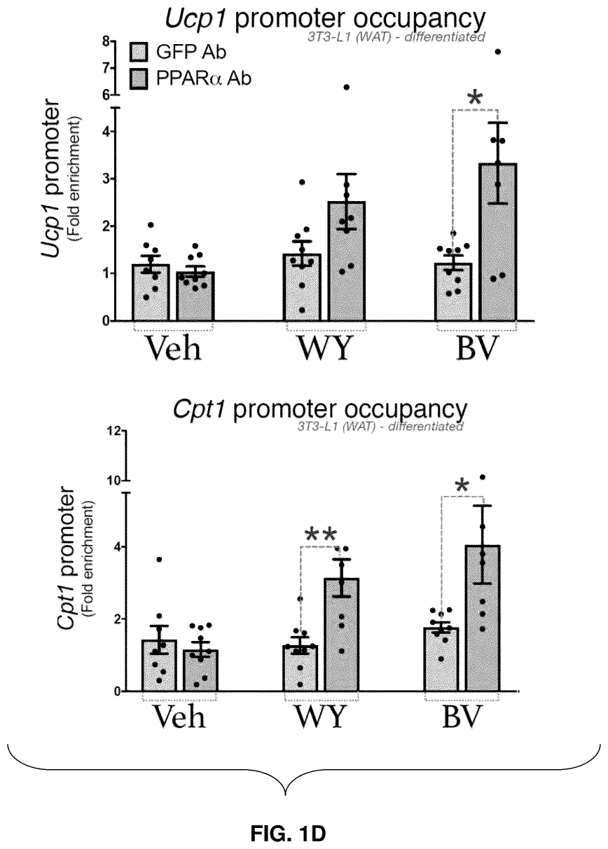

Whether bilirubin decreases adiposity by enhancing mitochondrial function was evaluated. It was previously shown that bilirubin reduces lipid accumulation in adipocytes. However, it remained to be determined if this occurs by activation of PPARα to reduce adiposity, selective actions on PPARγ, or is a dual PPAR agonist, which together may mediate its glucose- and lipid-lowering effects. First, to determine if bilirubin enhances genes for mitochondrial function, 3T3-L1 cells, a WAT-type murine pre-adipocyte cell line that differentiates to full adipocytes, were treated with increasing concentrations of biliverdin, which is more soluble and is rapidly produced to bilirubin, over the 9-day adipocytic differentiation protocol. Increasing biliverdin treatments significantly reduced lipid accumulation at 10 μM and 50 μM (FIG. 1A). The highest level of biliverdin (50 μM) substantially (p=0.0659) decreased lipid accumulation, and significantly increased mitochondrial and lipid burning genes Ucp1 and Cpt1 mRNA expression (FIG. 1B). While 1 μM did not reduce (p=0.0659) lipid accumulation, it significantly heightened the mitochondrial gene Ucp1 mRNA but not Cpt1 expression.

Both PPARγ and PPARα have been shown to upregulate the expression of Ucp1. However, Cpt1 is considered PPARa-dependent, indicating that bilirubin may function in a PPARa-dependent mechanism. To compare the effects of bilirubin on the activation of PPARα or PPARγ on mitochondrial function, a SeaHorse XFe96 Analyzer was used to measure oxygen consumption rate (OCR) in fully differentiated 3T3-L1-WAT adipocytes treated with 50 μM biliverdin, 50 μM WY 14,463 (PPARα agonist), or 10 μM rosiglitazone (PPARγ-agonist). It was found that biliverdin and WY 14,643 significantly increased the mitochondrial OCR for maximum respiration (FIG. 1C). Biliverdin significantly elevated ATP production, which was not observed with rosiglitazone or WY 14,643. Rosiglitazone, but not biliverdin or WY 14,643, enhanced the coupling efficiency. None of the ligands affected non-mitochondrial respiration, basal respiration, or proton leak. These results indicate that bilirubin function is more like a PPARα ligand.

To further investigate if bilirubin is driving PPARα to heighten Ucp1 and Cpt1 to improve mitochondrial function, fully differentiated 3T3-LI WAT adipocytes were treated with 50 μM biliverdin or 50 μM WY 14,643 for 24 hours, and then chromatin immunoprecipitation (ChIP) was performed with an antibody specific to PPARα or control for green fluorescent protein (GFP). In FIG. 1D, it is seen that biliverdin significantly increased PPARα occupancy at the 13K enhancer of the Ucp1 and the −3306 to −3109 region of the Cpt1 promoter. WY 14,643 stimulated PPARα occupancy at both promoters, but only significantly higher at the Cpt1 promoter. These results indicate that bilirubin has a hormonal function to induce PPARα occupancy at Ucp1 and Cpt1 promoters to drive expression, and is not a ligand for PPARγ, which overall enhances mitochondrial function in white adipocytes.

### Bilirubin Selectively Modulates PPARα to Increase Mitochondrial Activity

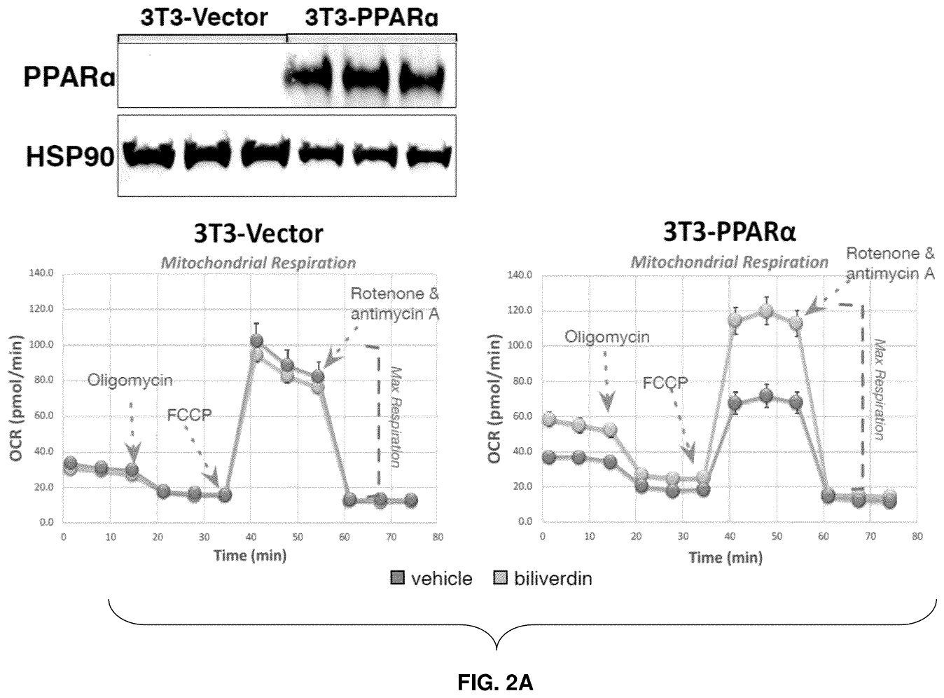

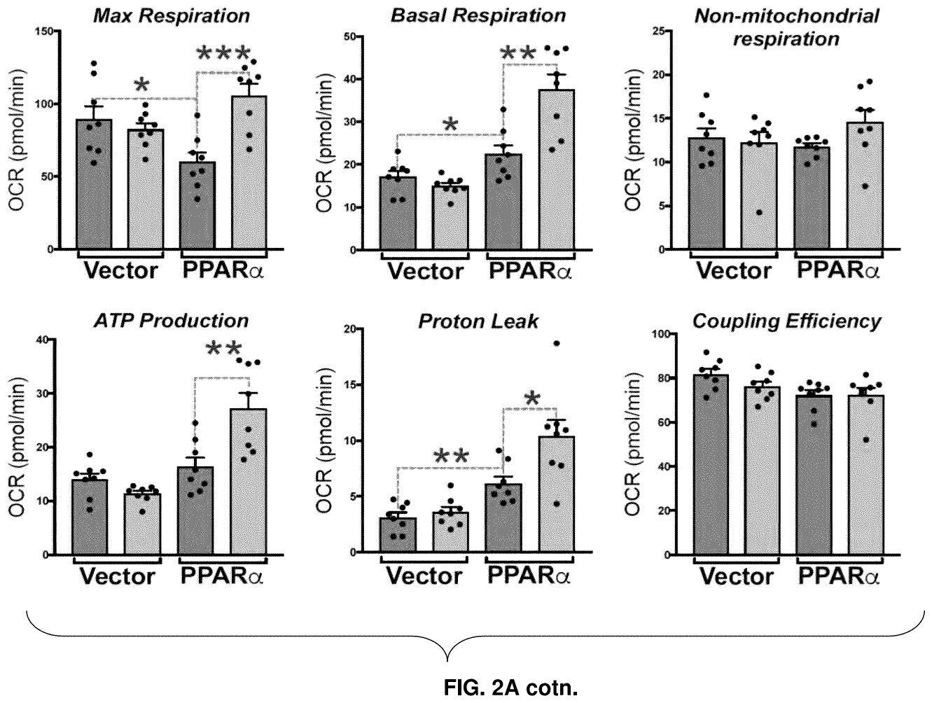

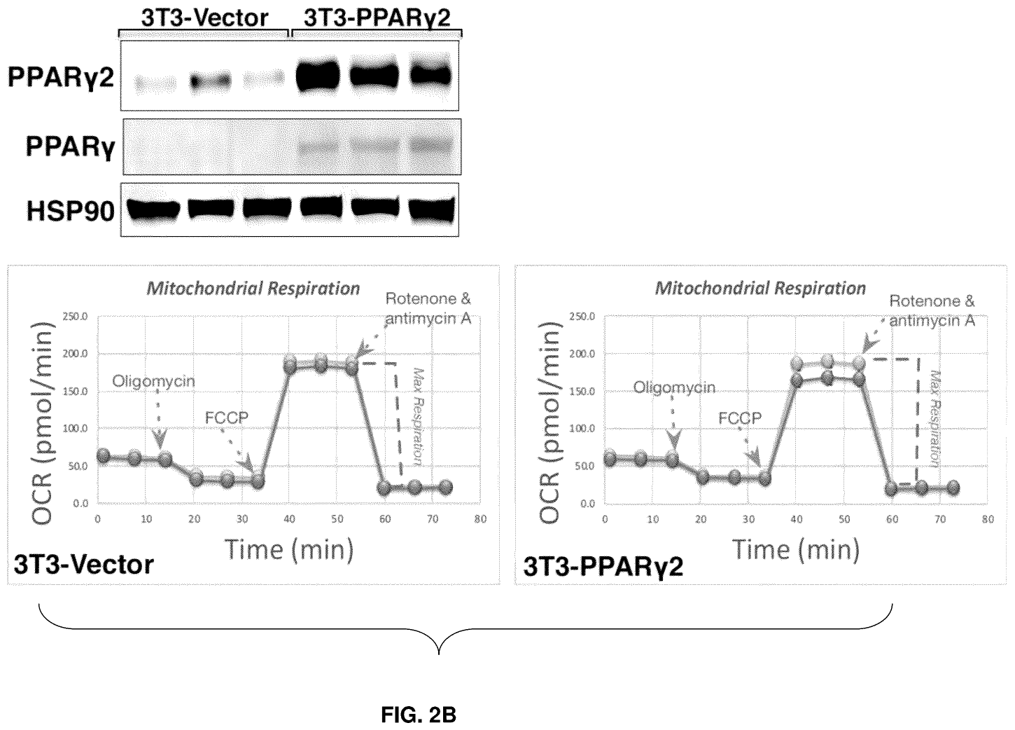

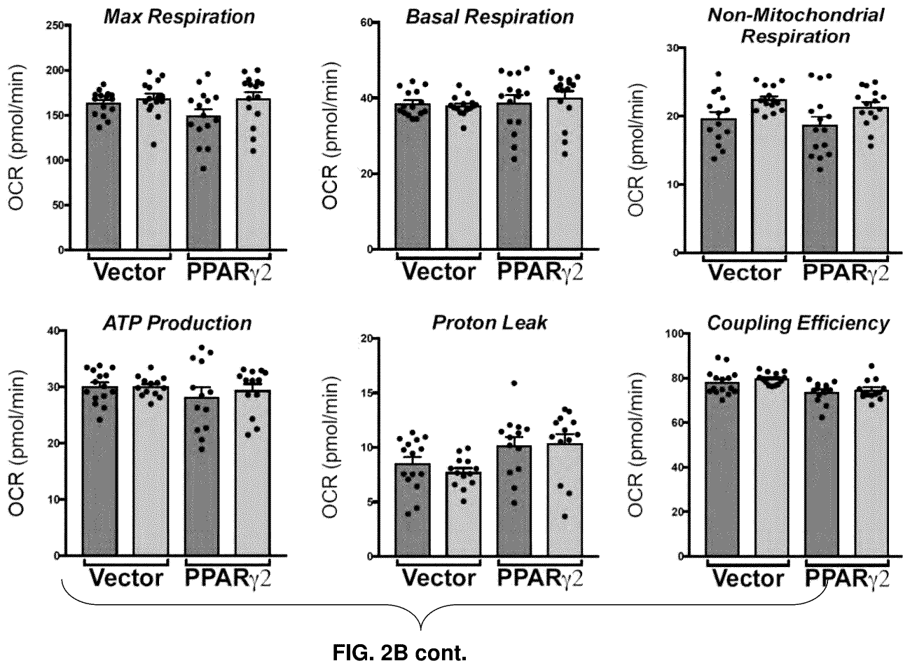

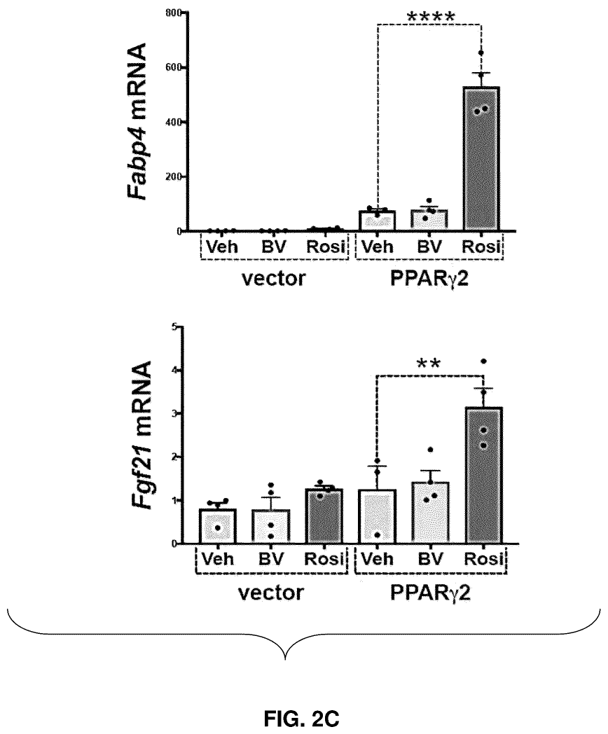

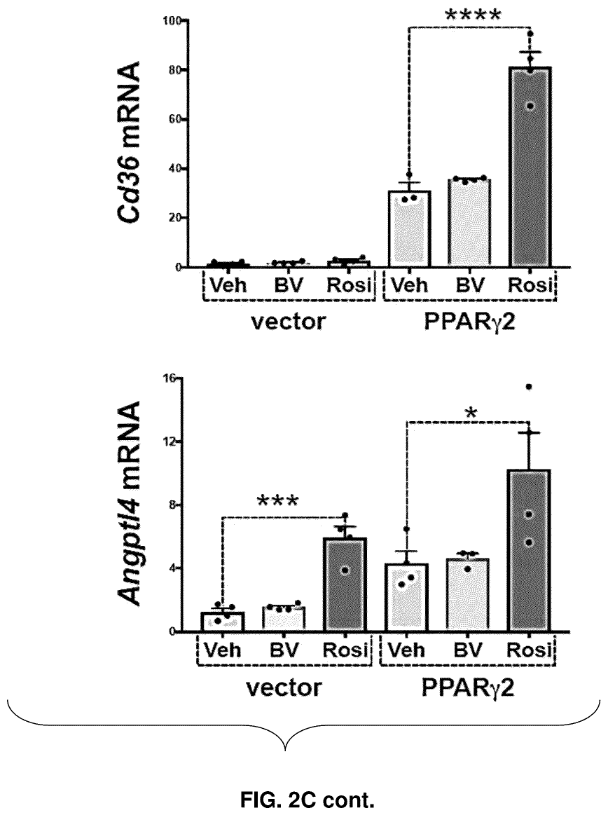

To investigate the specific role of bilirubin on PPARα or PPARγ as well as mitochondrial function and gene regulation, 3T3-LI cells that overexpressed each receptor (3T3-PPARα or 3T3-PPARγ2) were generated via lentivirus (FIGS. 2A-2B). As controls, lentiviral empty vector infected 3T3-L1 cells (3T3-Vector), which have very low or do not express the receptors in the undifferentiated state, were used. The 3T3-Vector cells had no responses to biliverdin in FIGS. 2A-2B. The 3T3-PPARα cells had significantly higher basal respiration and proton leak and lower maximum respiration. Biliverdin treatments in the 3T3-PPARα cells caused significantly higher maximum respiration, basal respiration, proton leak, and ATP production (FIG. 2A). Interestingly, the 3T3-PPARγ2 cells had no significant changes in mitochondrial respiration with biliverdin treatments. 3T3-PPARγ2 did not have significant increase in OCR (FIG. 2B) or gene related activity (FIG. 2C), which is consistent with bilirubin working through PPARα and not PPARγ (which causes weight gain and cardiovascular disease).

### Bilirubin Impacts Mitochondrial Function in Brown Adipocytes but not Lipid Levels

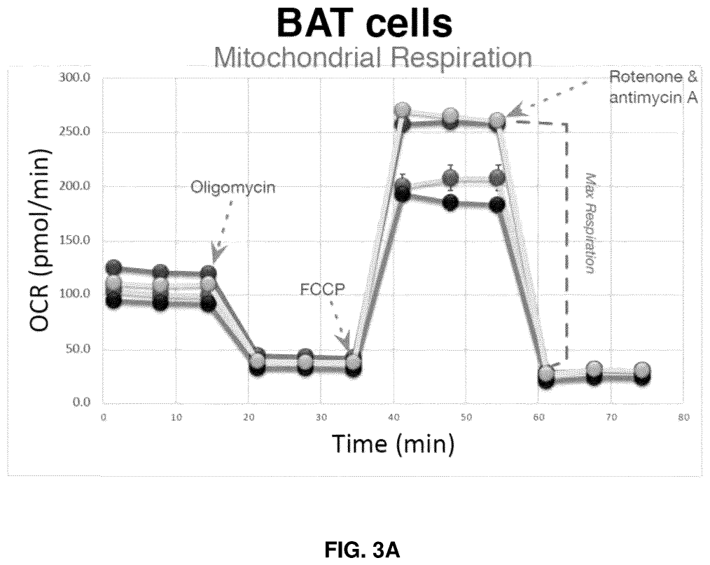

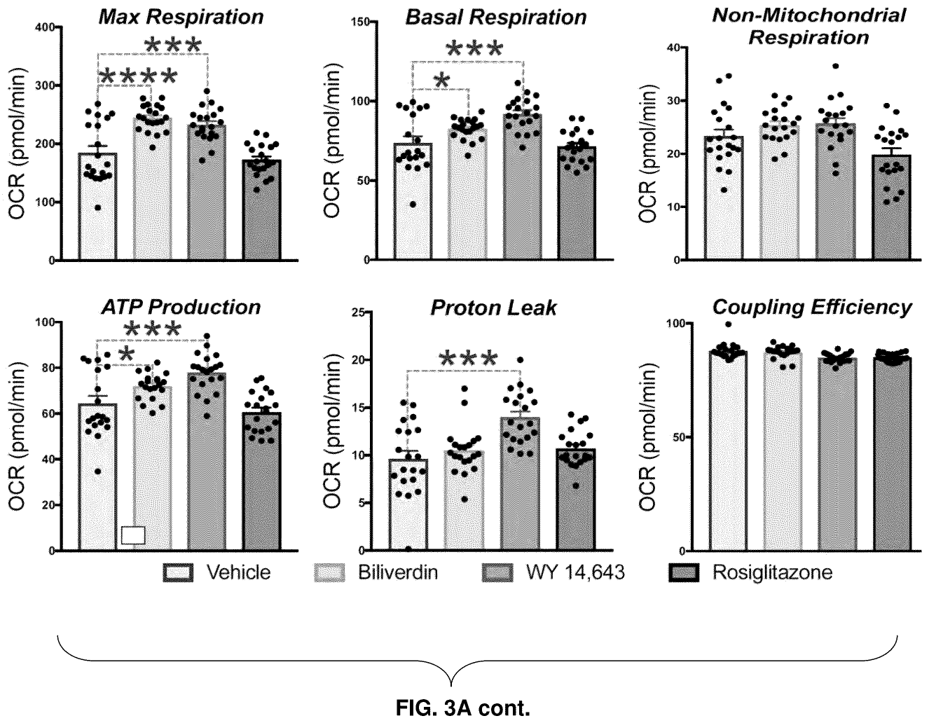

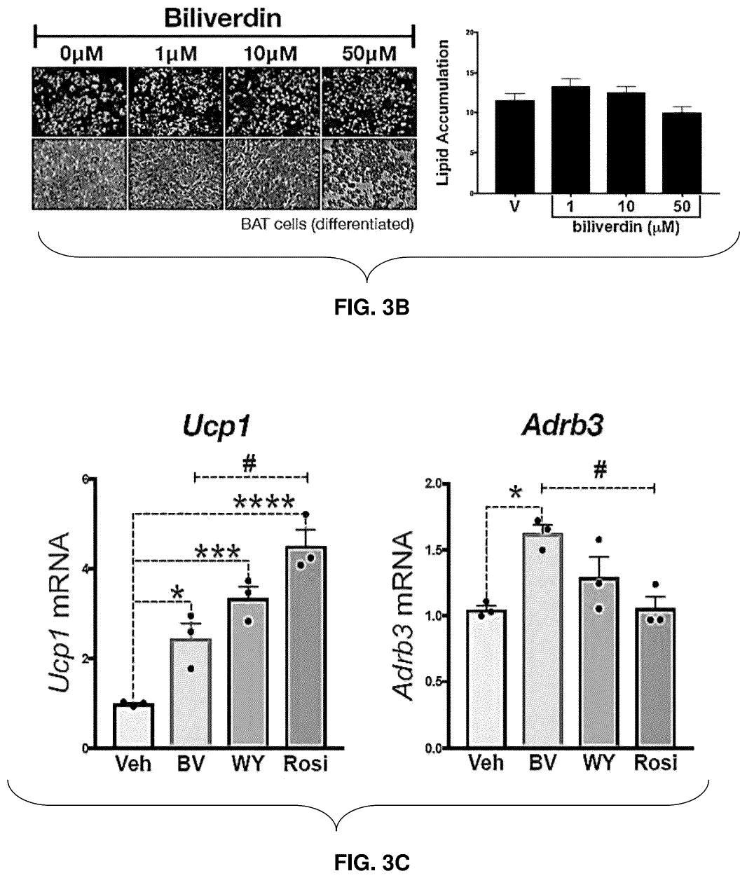

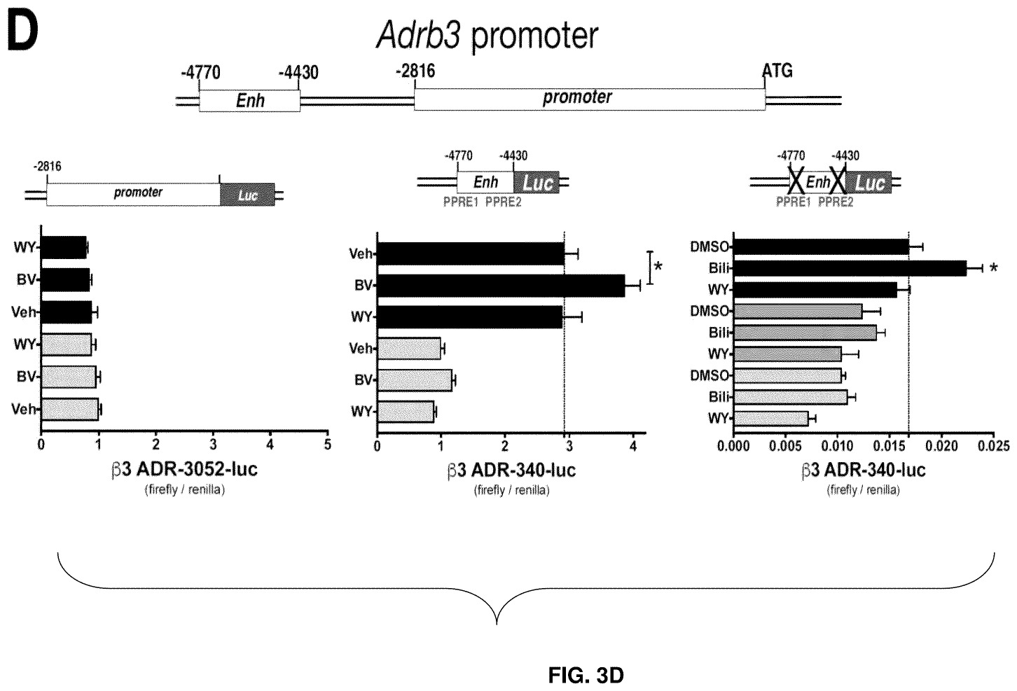

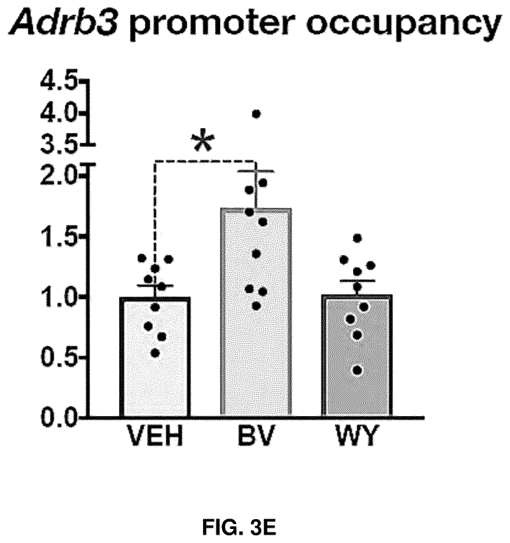

Energy expenditure was evaluated by SeaHorse analysis in a murine BAT cell line treated with biliverdin, rosiglitazone, WY 14,643 (FIG. 3A). The WY 14,643 and biliverdin treatments had similar results to the 3T3-L1 WAT model in that maximum respiration was significantly higher. Also, in the BAT cells, WY 14,643 and biliverdin treatments stimulated ATP production and proton leak. Rosiglitazone did not affect mitochondrial function in the BAT cells. Interestingly, increasing doses of biliverdin over the differentiation of the BAT cells had no impact on lipid accumulation (FIG. 3B). Treatment with 50 M biliverdin, 50 μM WY14,463, or 10 μM rosiglitazone in differentiated BAT cells for 24 hrs caused a significant increase in Ucp1 mRNA with all three ligands (FIG. 3C). However, B3 adrenergic receptor (Adrb3) was only significantly higher with biliverdin, which is known for its excitation of BAT thermogenesis. PPARα has been previously shown to upregulate Adrb3 and Ucp1 to induce the browning of adipocytes and improve mitochondrial function. PPARγ was shown to lessen Adrb3 expression in adipocytes causing lipogenesis.

To determine if biliverdin/bilirubin is increasing Adrb3 in a PPARa-dependent manner, the proximal (−2816 to +118) and enhancer (−4770 to −4430) regions of the murine promoter in the pGL4.10 construct were cloned. The constructs were transfected with or without PPARα in receptorless Cos7 cells. The proximal promoter had no response with or without PPARα expressed in Cos7 cells (FIGS. 3D-3E). However, there was a significant increase with PPARα overexpression with the enhancer region which was significantly higher with biliverdin treatment. Two PPAR response elements (PPREs) were identified in the enhancer region, and mutations in each separately caused no induction of luciferase activity with biliverdin. There have been no PPREs identified in the Adrb3 promoter, even though PPARα has been shown to heighten its expression. Therefore, various areas within the Adrb3 promoter were analyzed using the information from the luciferase promoter data and with analysis of several suspected PPREs in the promoter region. It was found that the PPRE in the enhancer region of the Adrb3 gene had the highest predicted PPAR binding.

To determine if bilirubin is driving PPARα to the Adrb3 promoter at the enhancer region, ChIP was performed with an antibody specific to PPARα (described above) with biliverdin and WY 14,643 treatments. Biliverdin intensified the occupancy of PPARα at the enhancer region of the Adrb3 promoter (FIG. 3F). Similar to the mRNA and luciferase promoter responses, WY 14,643 did not increase the occupancy of PPARα to the Adrb3 promoter. WY 14,643 and biliverdin increased the occupancy of PPARα at 13K enhancer of the Ucp1 and the Cpt1 promoters in BAT cells. These results show that the actions of bilirubin to improve mitochondrial function are selective and most likely PPARa-dependent.

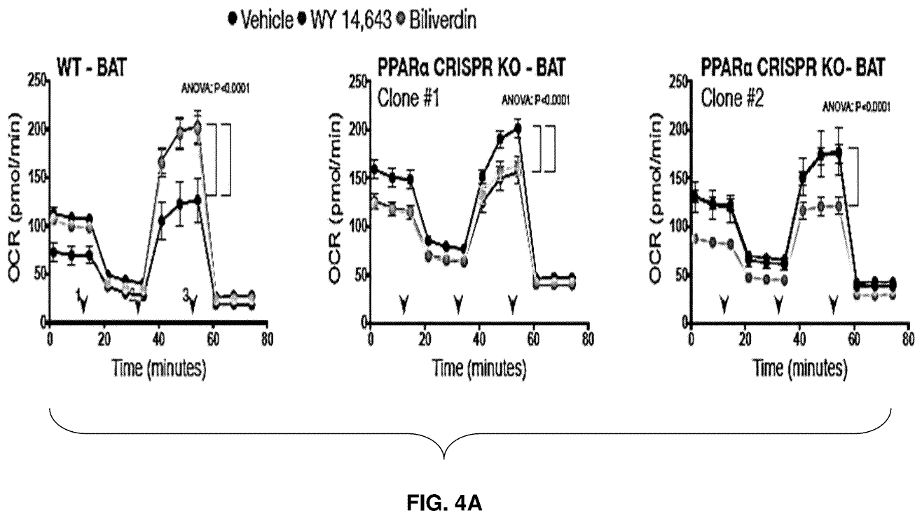

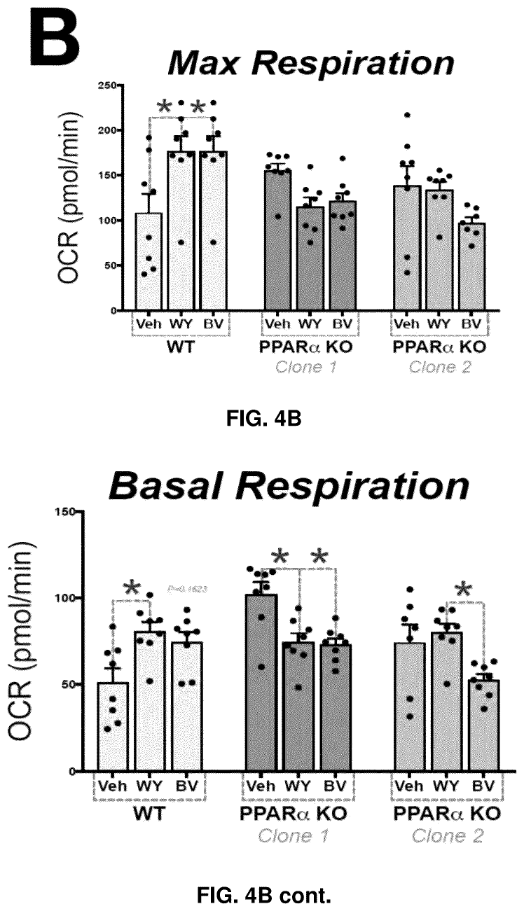

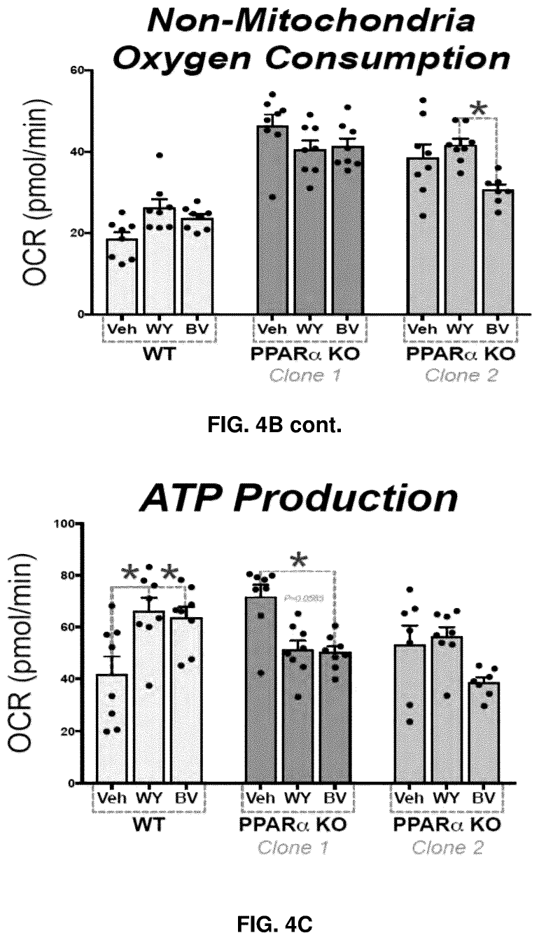

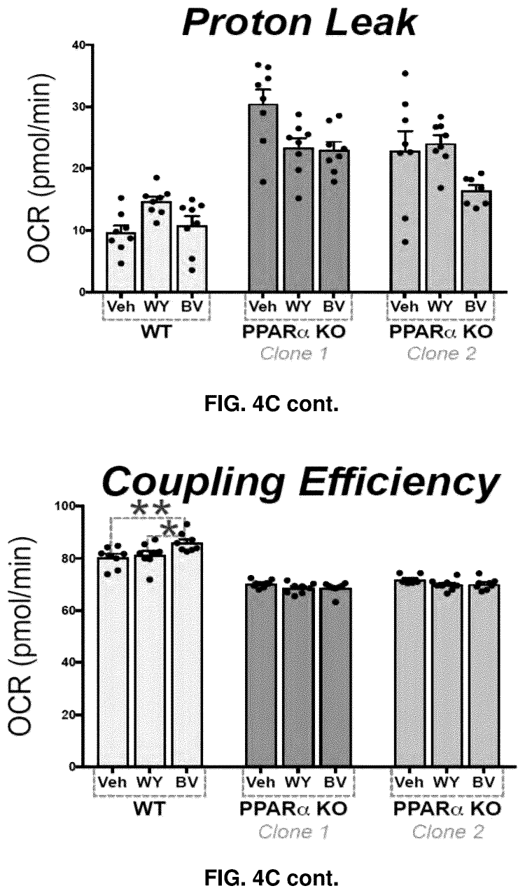

To further delineate the actions of bilirubin on BAT, CRISPR technology was developed to knockout (KO) PPARα and establish two null clone lines. The BAT PPARα CRISPR KO cells (clone 1 and 2) and wild-type (WT) cells were treated with biliverdin or WY 14,643 for 24 hours and the impact on mitochondrial function was determined via Seahorse analysis. The WT BAT cells responded as previously shown (FIG. 3A) with increased OCR with WY 14,643 and biliverdin for maximum respiration, basal respiration, and ATP production (FIGS. 4A-4B). The function of the ligands was lost in both clones for the BAT PPARα CRISPR KO cells.

### Bilirubin Induces a Selective Set of Co-Regulators to Bind PPARα

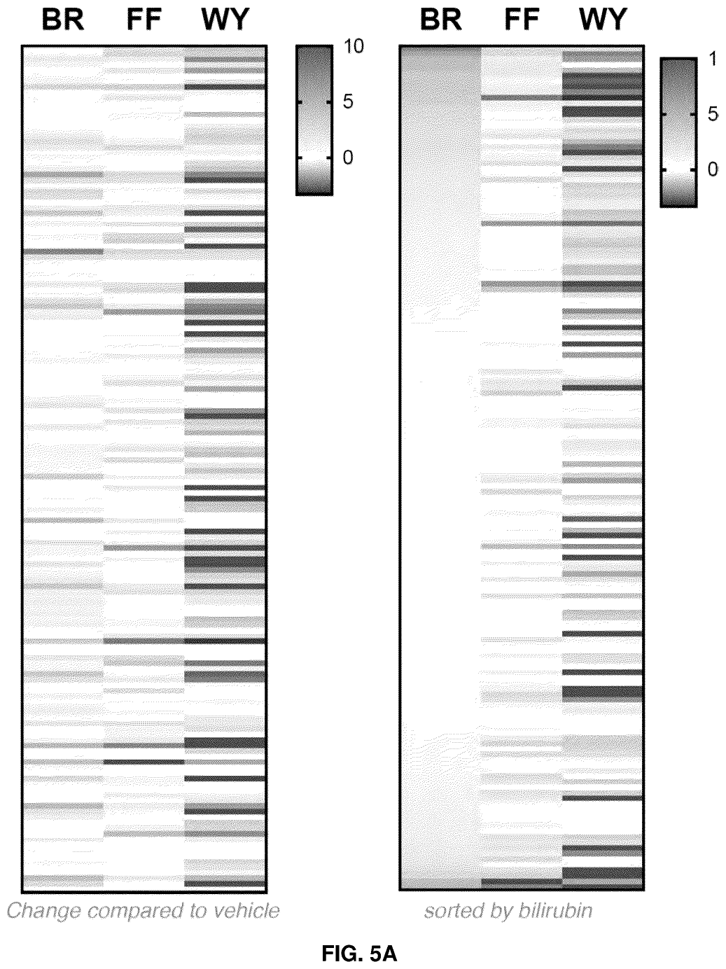

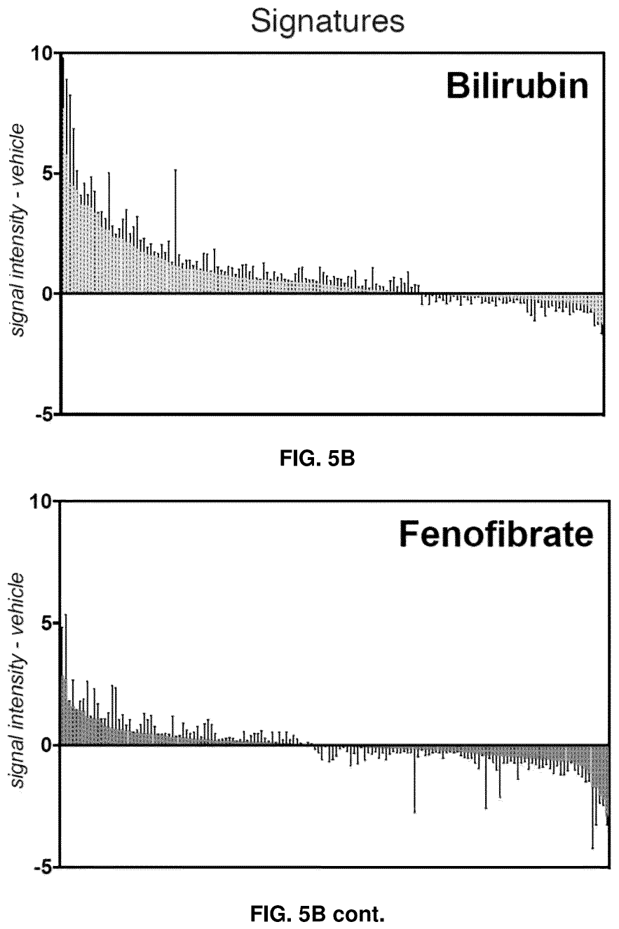

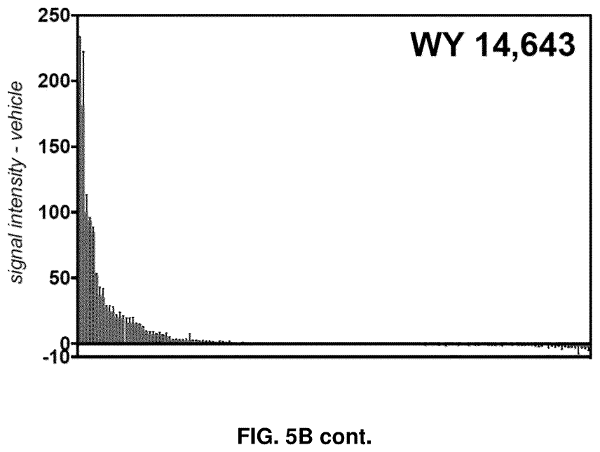

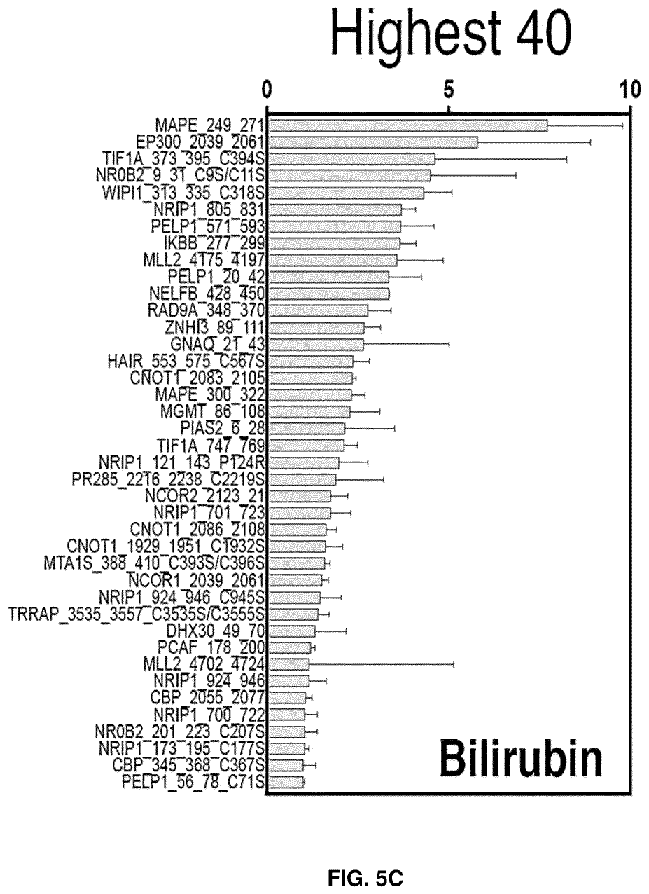

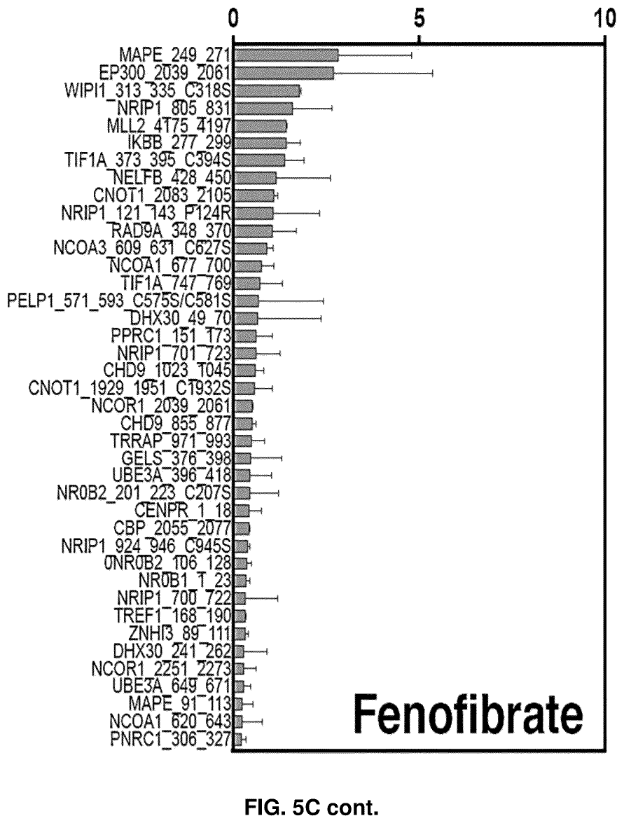

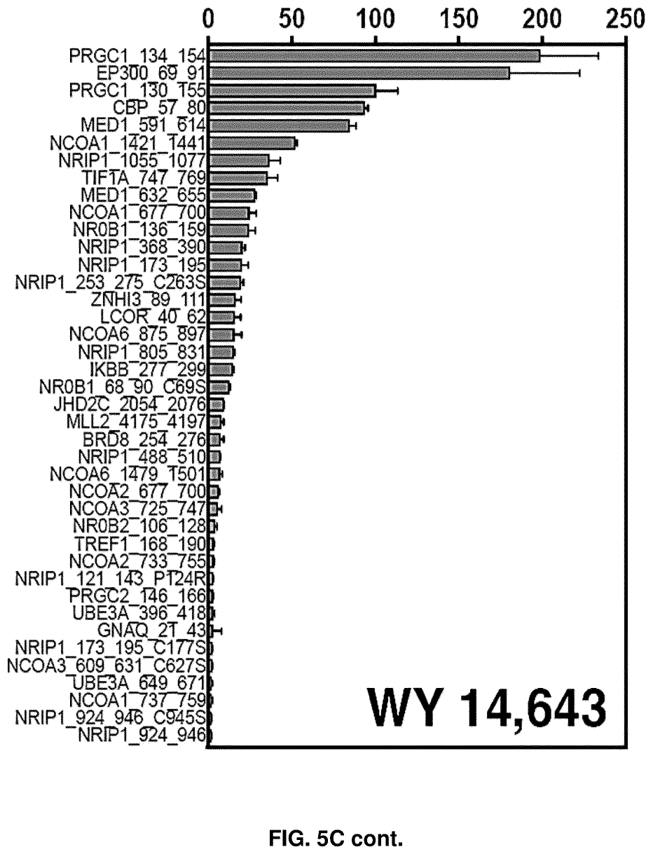

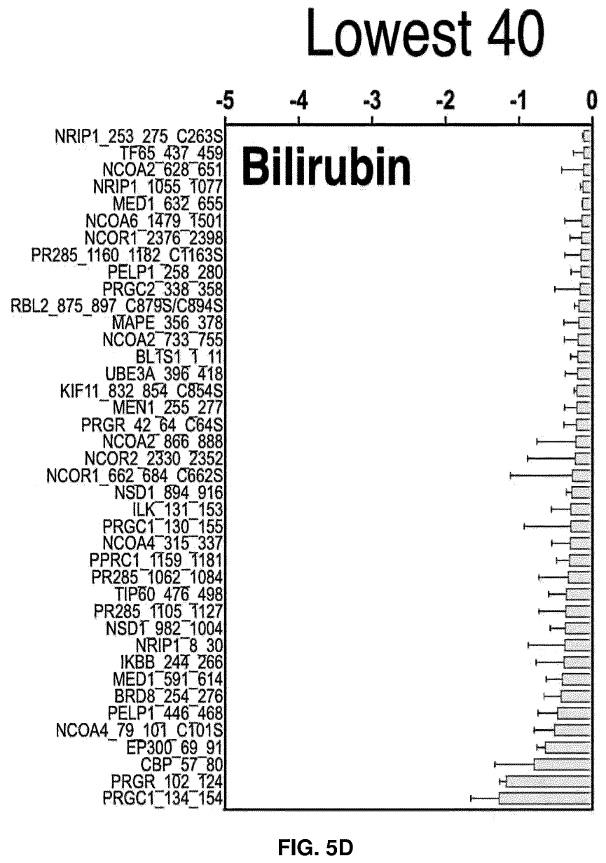

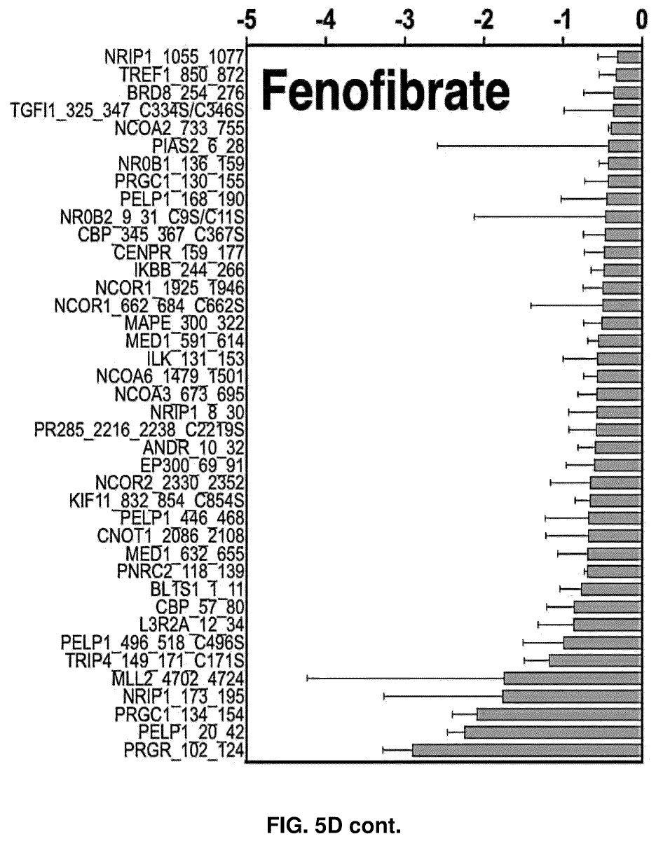

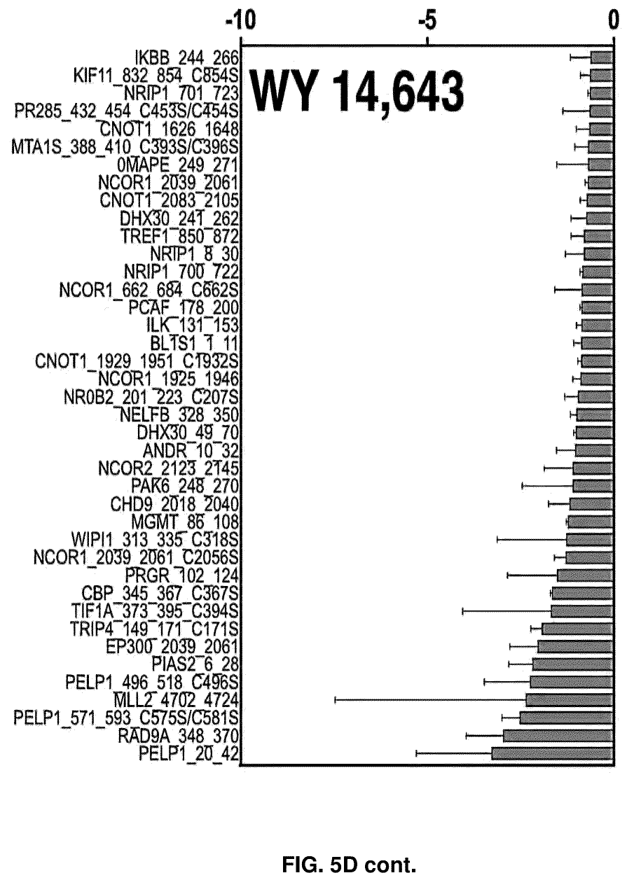

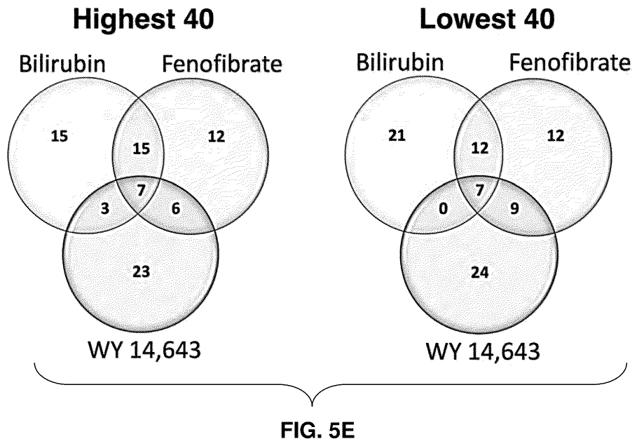

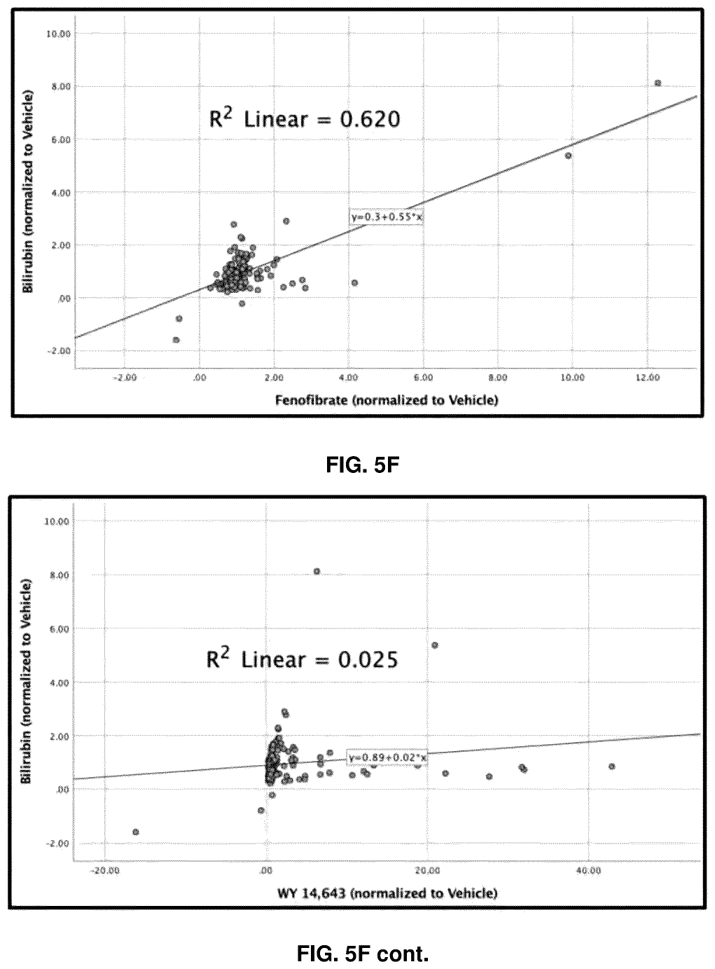

The molecular determinants that dictate specificity and selectivity in PPARα-coregulator interactions are largely unknown. PPARα ligands have different binding affinities, which may result in a slight conformational change in the protein that may lead to divergent PPARα transcriptional activity, which has been shown between fenofibrate and WY 14,643. To determine if bilirubin binds to the ligand binding domain (LBD) of PPARα to cause recruitment of a specific set of co-regulator proteins, the Microarray Assay for Realtime Coregulator-Nuclear Receptor Interaction (MARCONI) technology was used. The purified human PPARa-LBD was used in solution to determine if bilirubin directly interacts and how PPARα responds to coregulator recruitment compared to synthetic PPARα ligands fenofibrate and WY 14,643. The ligand was applied to the human PPARa-LBD in solution on the MARCONI nuclear hormone receptor (NHR) chip to systematically characterize the binding between ligands with the human PPARa-LBD, and how this affects PPARα binding with 154 coregulator motifs. In FIG. 5A, it is shown that bilirubin, fenofibrate, and WY 14,643 mitigate binding of the human PPARα LBD to coregulator motifs. Sorting of bilirubin from highest to lowest coregulator binding (FIG. 5A-left) shows that fenofibrate has comparable coregulator recruitment, but WY 14,643 has a distinct coregulator recruit that is much different compared to bilirubin or fenofibrate. The molecular signatures of bilirubin and fenofibrate were also similar (FIG. 5B). However, WY 14,643 showed a significant different molecular fingerprint compared to the other two ligands. To identify common and unique coregulators between the ligands, the highest 40 and lowest 25 coregulator binding affinities subtracted from the vehicle were sorted to remove the background (FIG. 5C). Several highest interacting coregulators showed for bilirubin and fenofibrate such as MAPE (LXXL 249-271), WIPII (LXXL 313-335), CNOT1 (LXXL 2083-2105), PELP1 (LXXL 571-593), and others. However, these are not in the highest coregulators recruited to PPARa-LBD for WY 14,643 which was PRGC1 (LXXL 134-154), PRGC1 (LXXL 130-155), MED1 (LXXL 632-655), and CBP (LXXL 57-80). There were overlaps on high coregulator recruitment with all three ligands for TIF1A (LXXL 373-395), and EP300 for LXXL 2039-2061 for bilirubin and fenofibrate but LXXL 69-91 for WY 14,643. As for reduced interactions with the human PPARα LBD, bilirubin and fenofibrate showed that PRGR (LXXL 102-124), PRGC1 (LXXL 134-154), and PELP1 (LXXL 446-468). These coregulator interactions were not reduced with WY 14,643, but there were similarly reduced interactions with fenofibrate and WY 14,643 for MLL2 (LXXL 4702-4724) and TRIP4 (LXXL 149-171) but not bilirubin. These data show that bilirubin has direct binding to the human PPARa-LBD and induces coregulators and that some of them are also recruited by fenofibrate binding. WY 14,643 binding to the human PPARa-LBD causes a diverse group of coregulators compared to bilirubin and fenofibrate. The variances in coregulator recruitment may explain the differential in gene regulation and physiological responses with each ligand.

Obese Mice Treated with Bilirubin have Higher Mitochondrial Function in WAT by Enhanced Coregulator Recruitment to PPARα

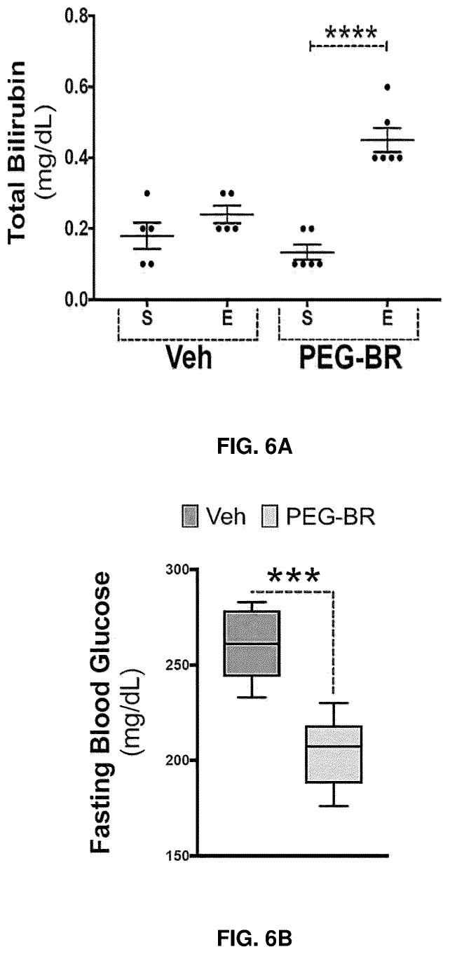

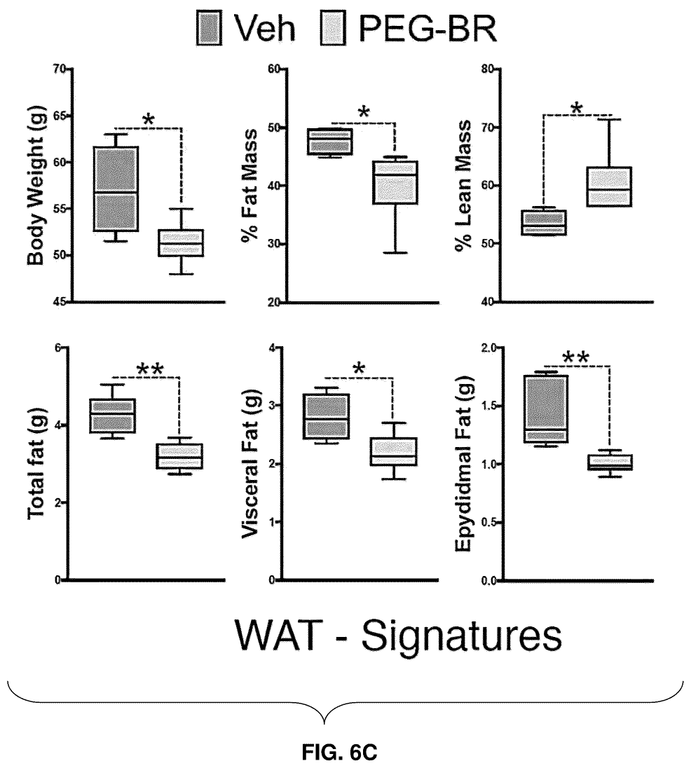

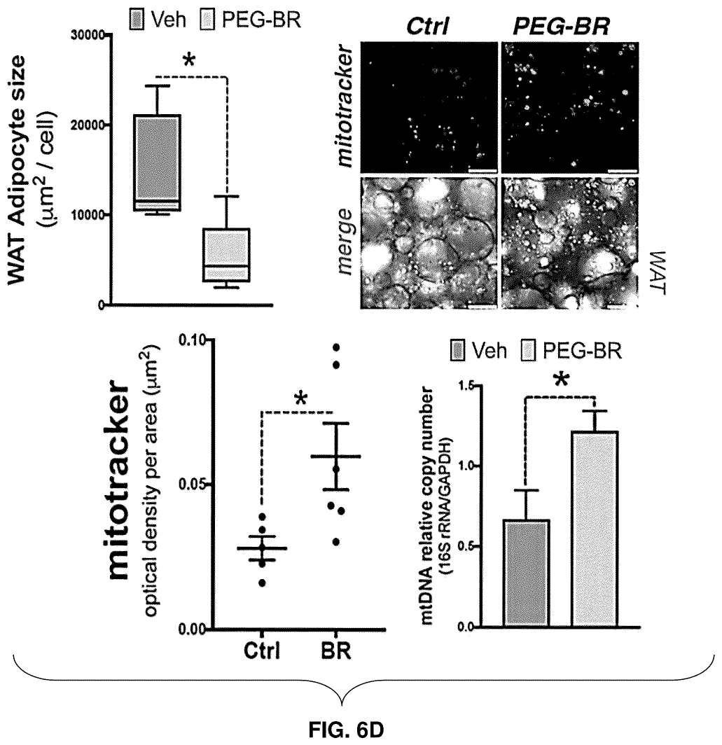

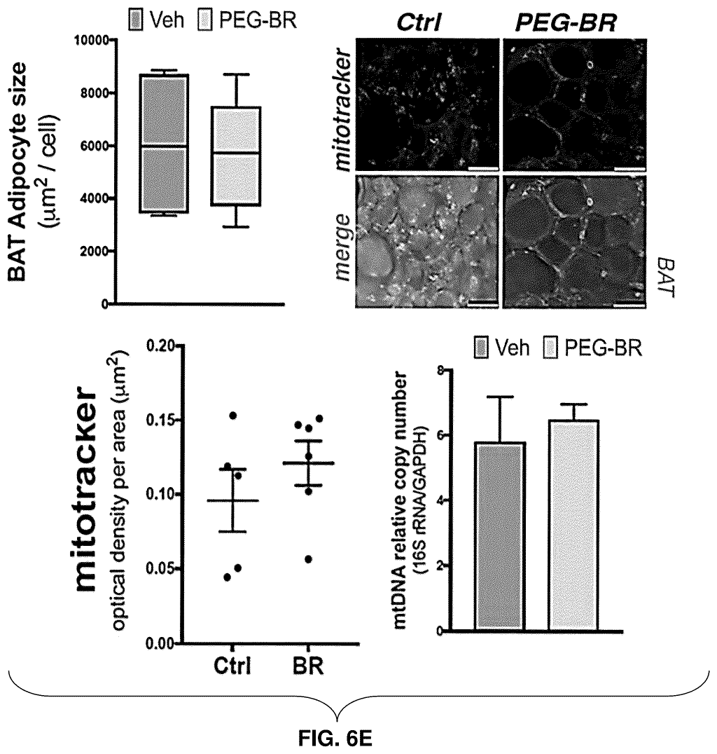

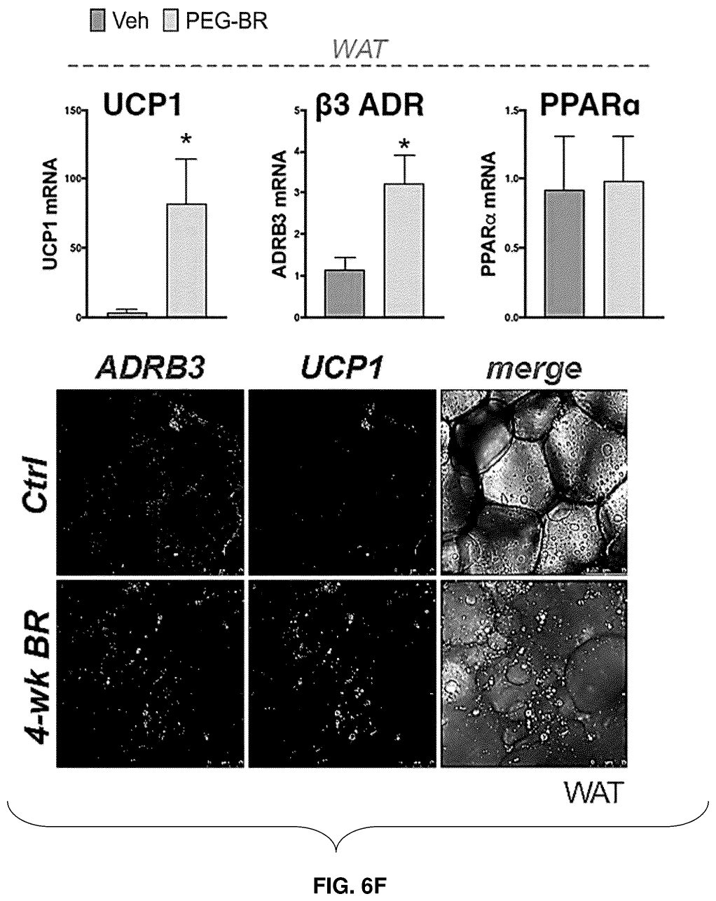

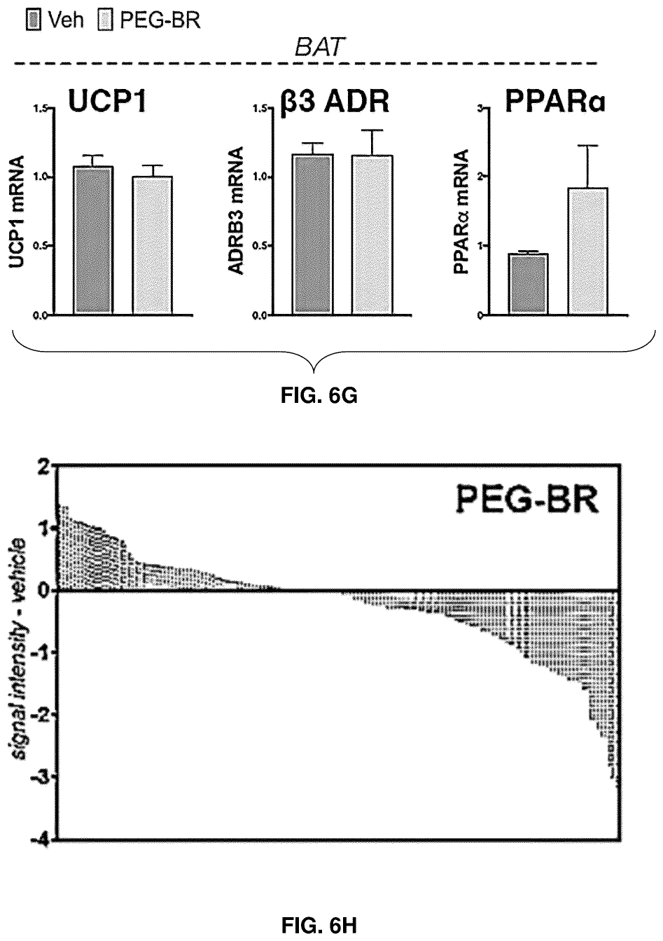

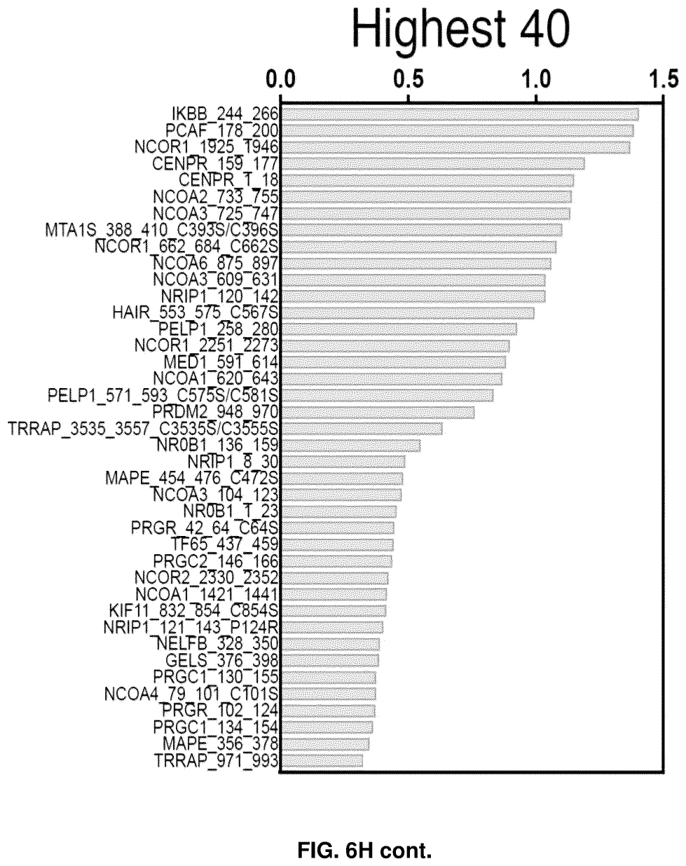

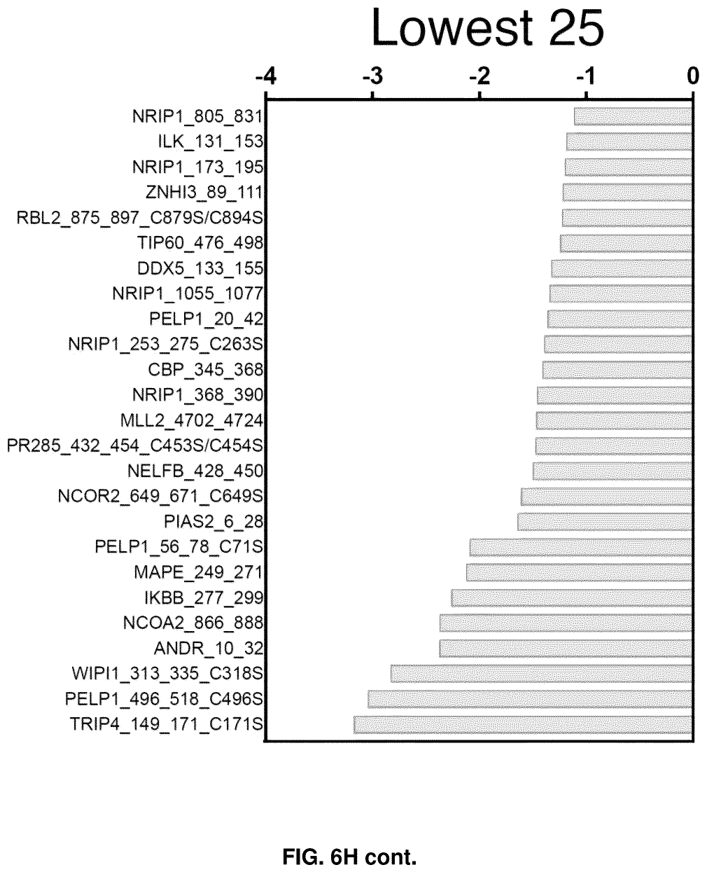

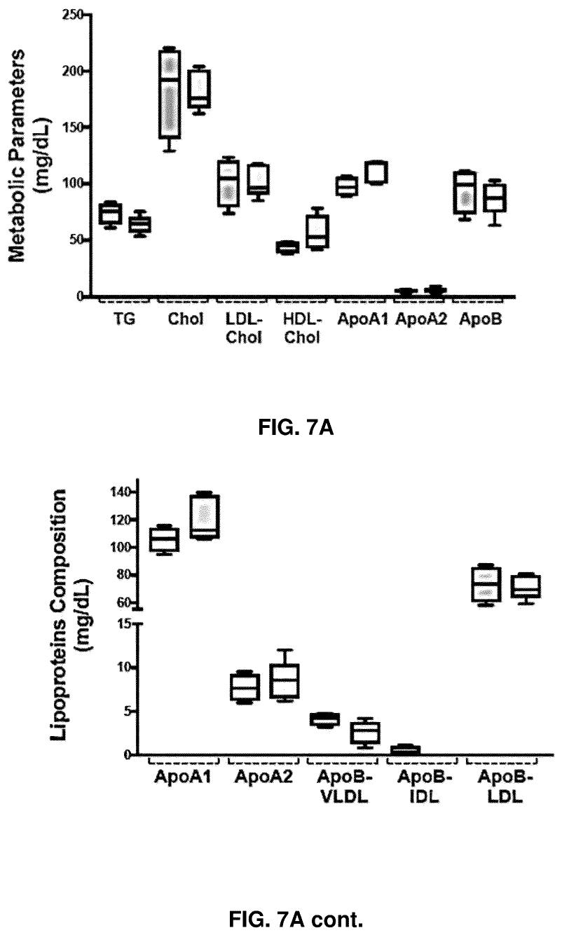

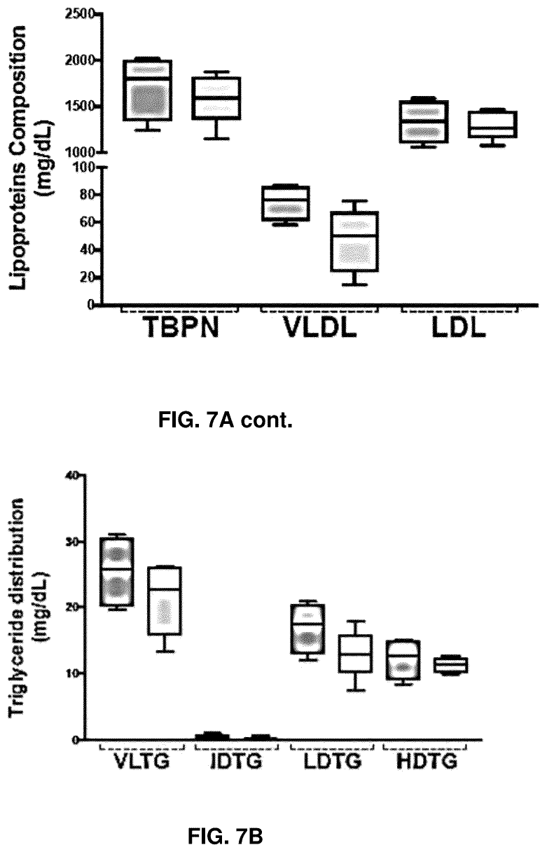

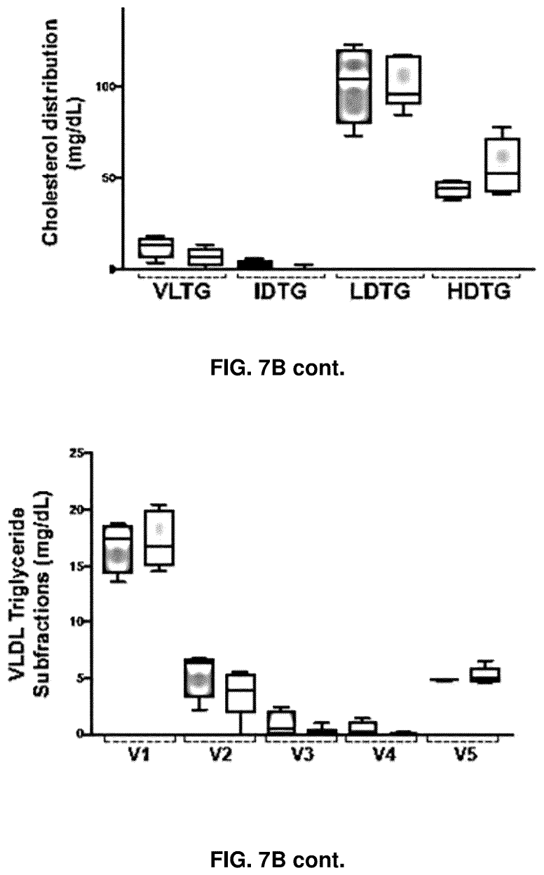

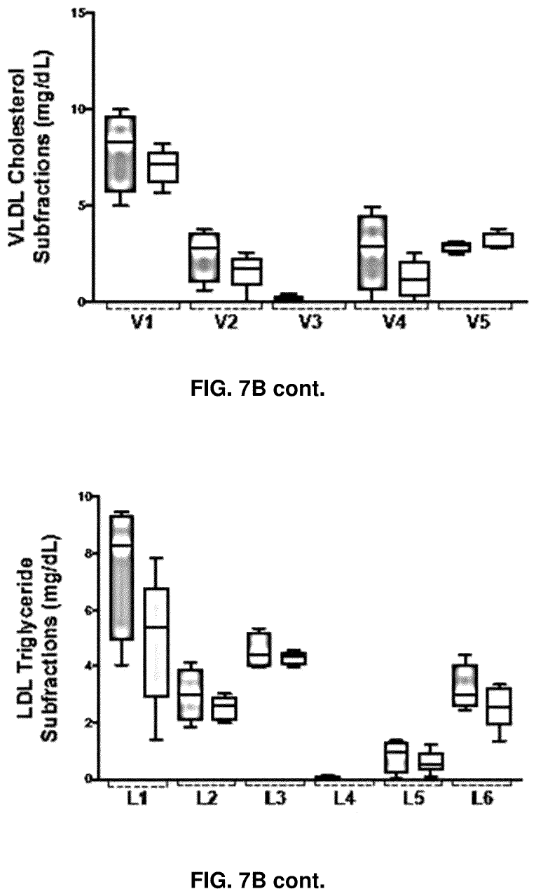

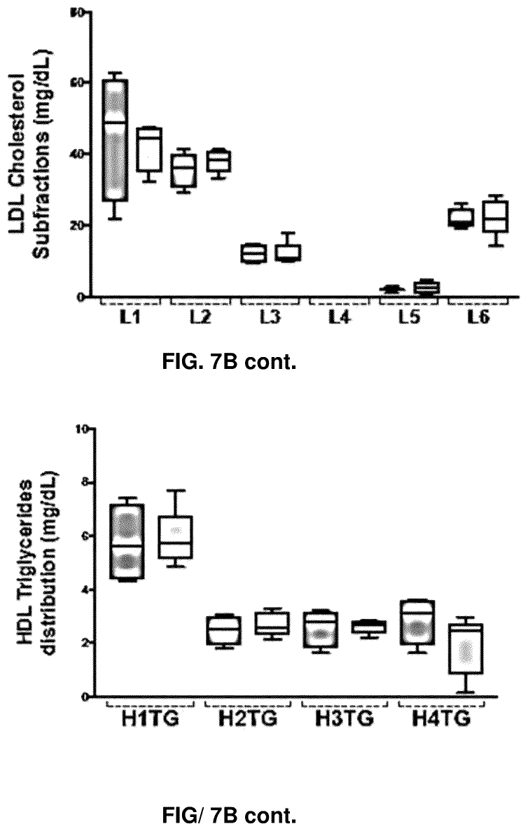

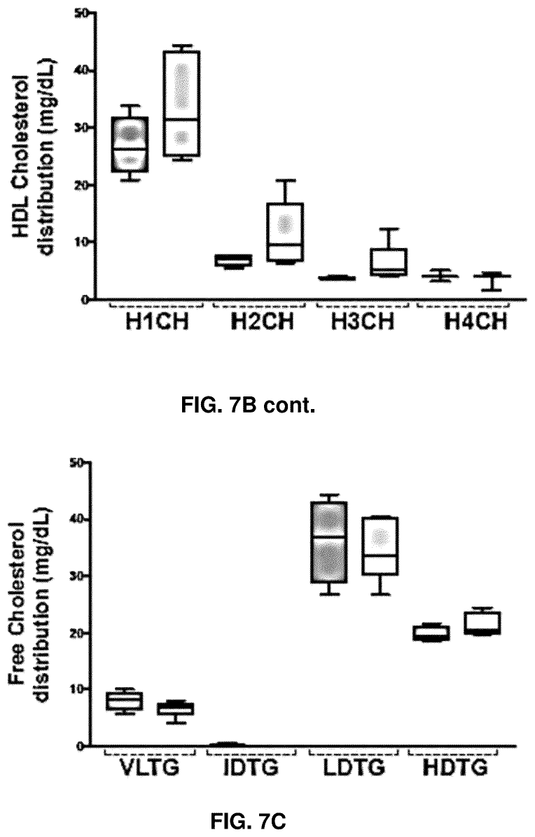

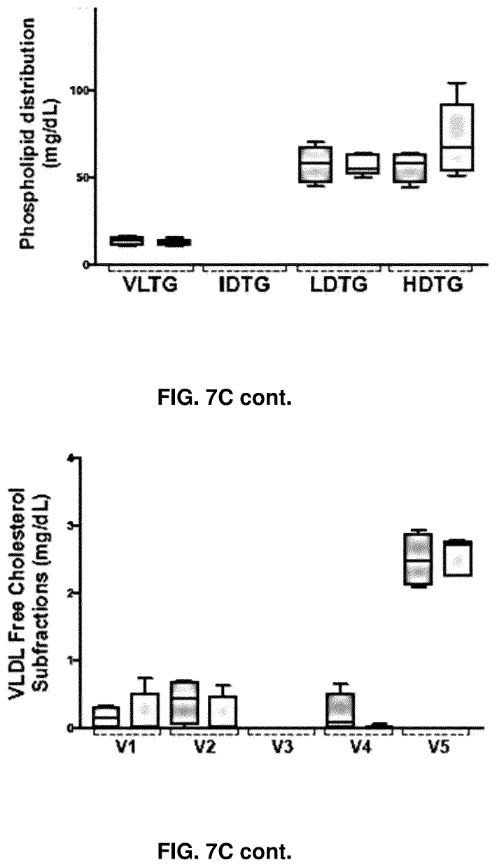

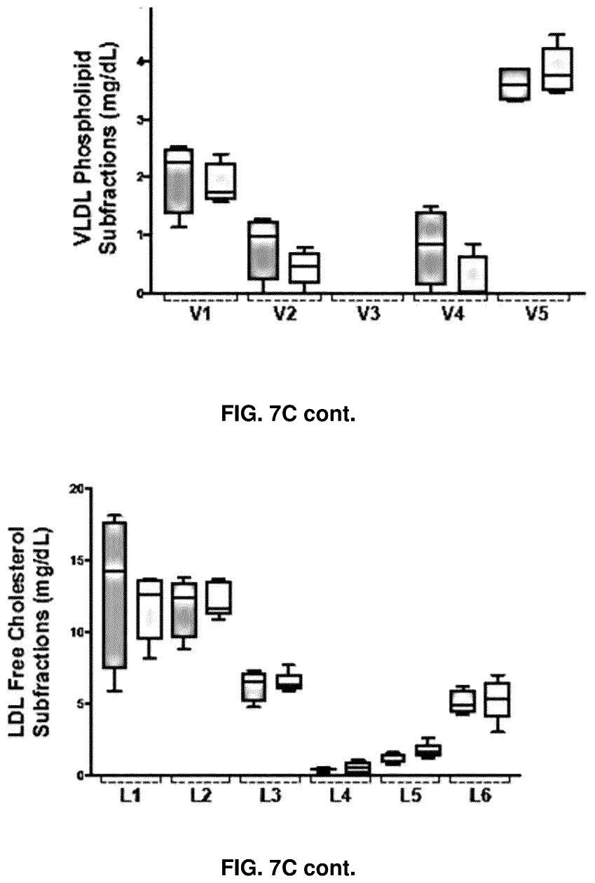

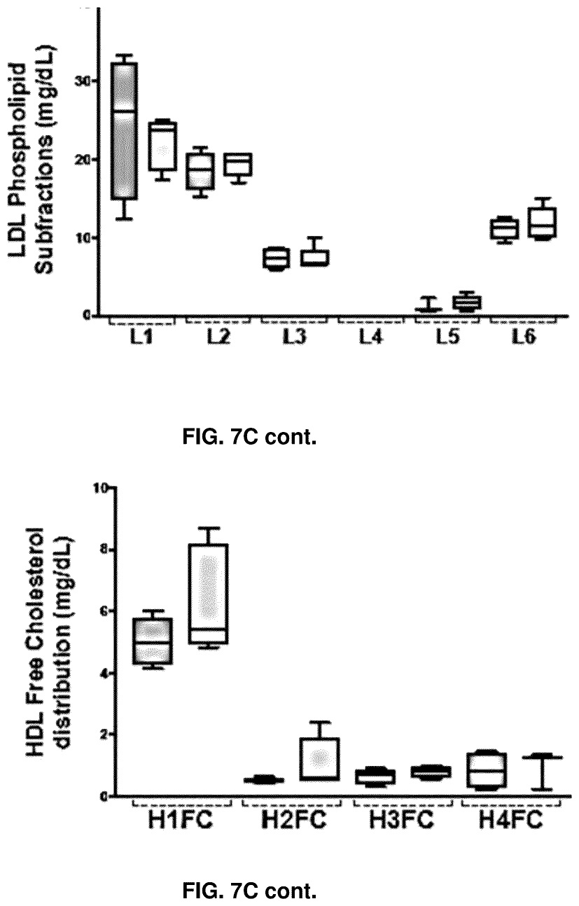

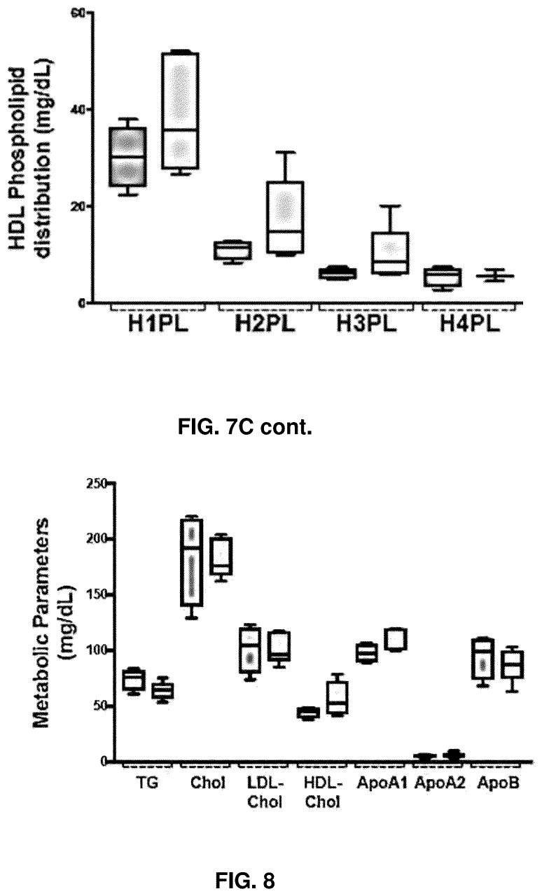

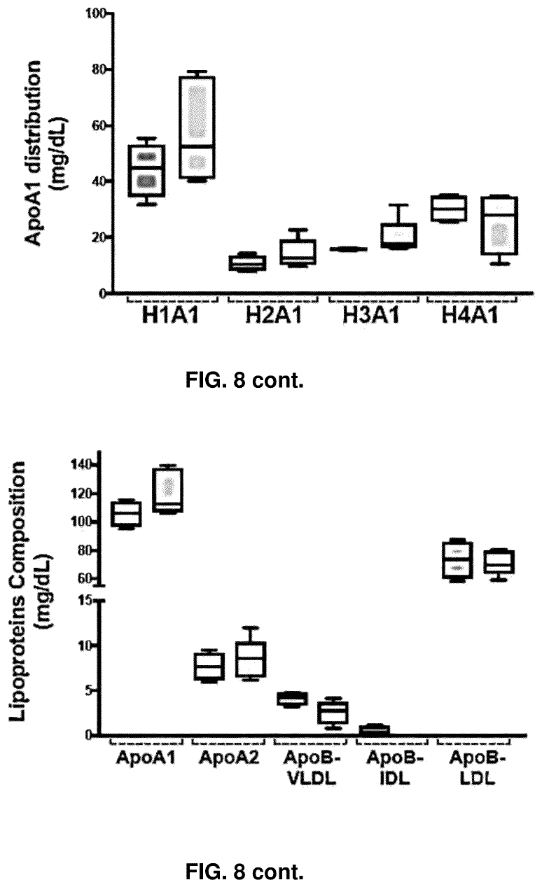

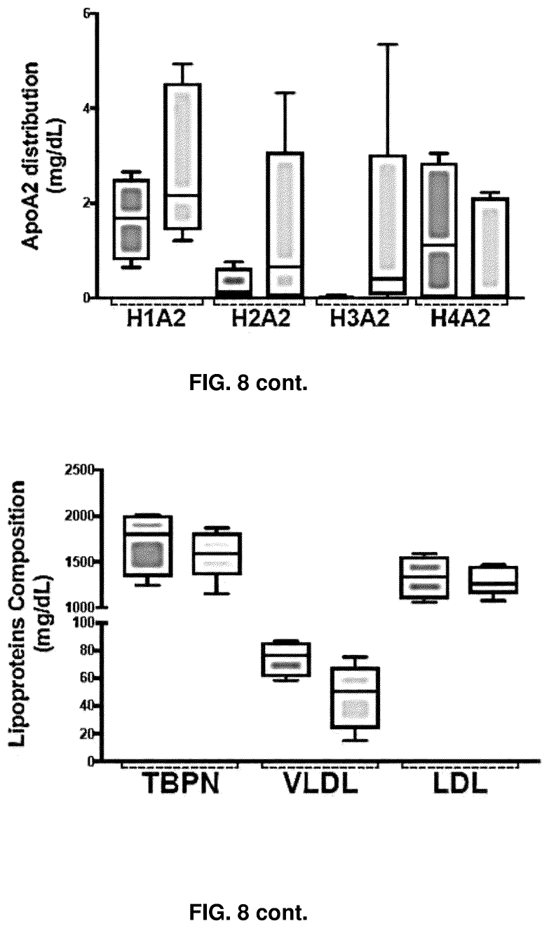

To determine if the lower adiposity in the hGS patients with hyperbilirubinemia is BR specific, and not due to only reduced UGT1A1 activity, diet-induced obese (DIO) mice were treated with water-soluble PEGylated BR (PEG-BR). In FIGS. 6A-6C, it is seen that a 4-wk treatment with PEG-BR in obese mice caused a significant reduction in blood glucose, weight gain, fat mass, and increased lean mass. The WAT size was significantly lower (p<0.05) and WAT mitochondrial function and number was higher (FIG. 6D). Interestingly, PEG-BR did not affect BAT size or BAT mitochondrial function or number (FIG. 6E). Measurement of fat burning genes Ucp1 and Adrb3 in WAT was higher (FIG. 6F), but not in BAT (FIG. 6G). There were also no significant changes in PPARα expression in WAT and BAT tissues (FIG. 6F & FIG. 6G). MARCONI nuclear hormone receptor analysis of endogenous PPARα in WAT of the obese mice treated with PEG-BR and vehicle revealed that PEG-BR induces a binding of coregulators and a unique molecular signature (FIG. 6H). The highest binding results of the MARCONI assay revealed that PEG-BR enhanced binding with several coregulators, most notable was several amino acids that are contained in nuclear receptor coactivators (NCOA2, NCOA3, NCOA6, NCOA1, and NCOA4), nuclear receptor corepressors (NCOR1 and NCOR2), and peroxisome proliferator-activated receptor gamma coactivator 1-alpha (PGC-1a) (FIG. 6H). The coregulators with reduced binding (lowest) showed that several proteins have lower interaction to PPARα with PEG-BR treatments, with five sites with reduced interaction for nuclear receptor interacting protein 1 (NRIP1, also known as RIP140). These data show that PEG-BR induces a specific set of coregulators to bind PPARα that regulates WAT size and increases mitochondrial function and number. Furthermore, FIGS. 7-8 show that PEG-BR reduces plasma triglycerides, VLDL, ApoB-VLDL, and LDL cholesterol, and increases ApoA1, and HDL cholesterol.

High-Fat Fed Mice with Hyperbilirubinemia are Resistant to WAT Hypertrophy by Enhanced Coregulator Recruitment to PPARα

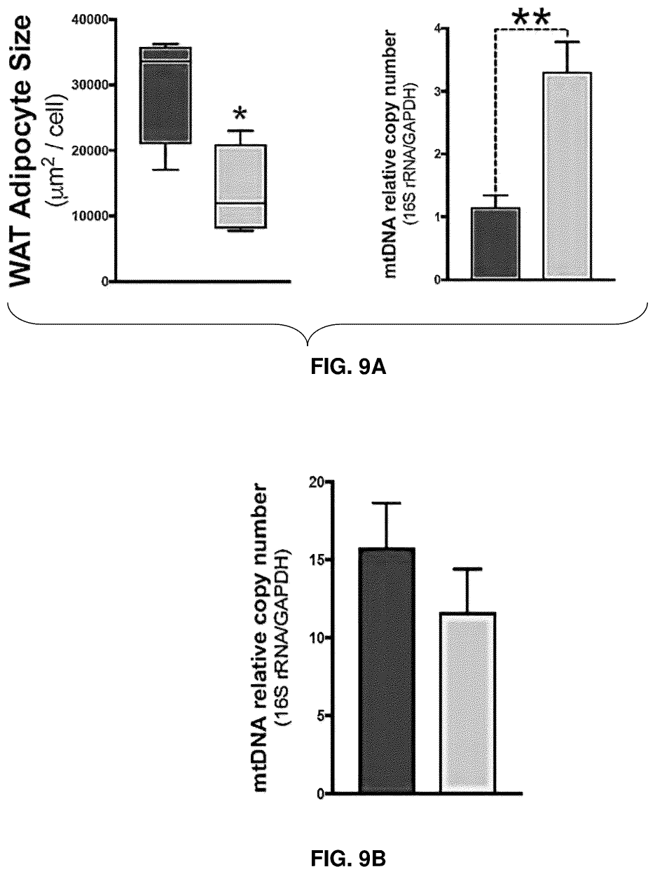

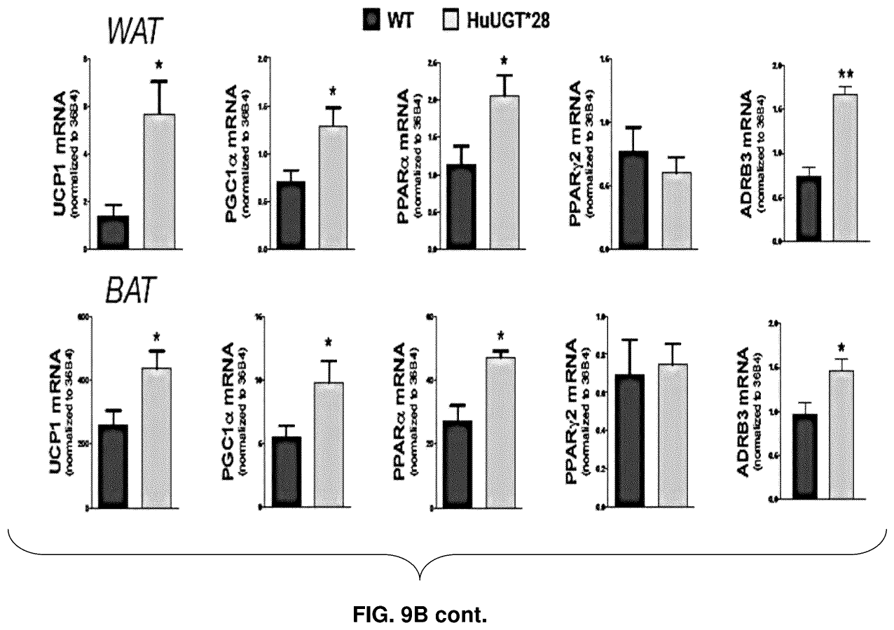

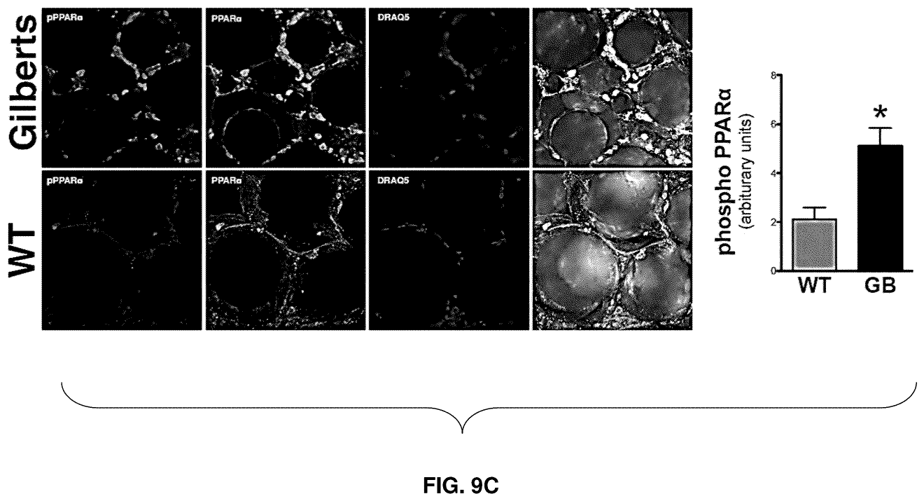

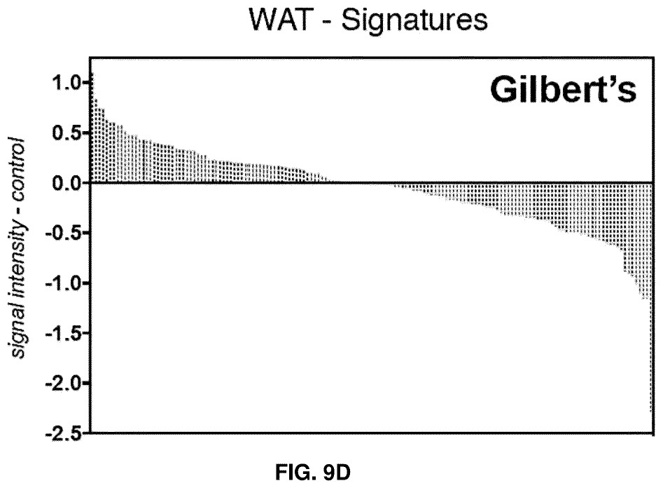

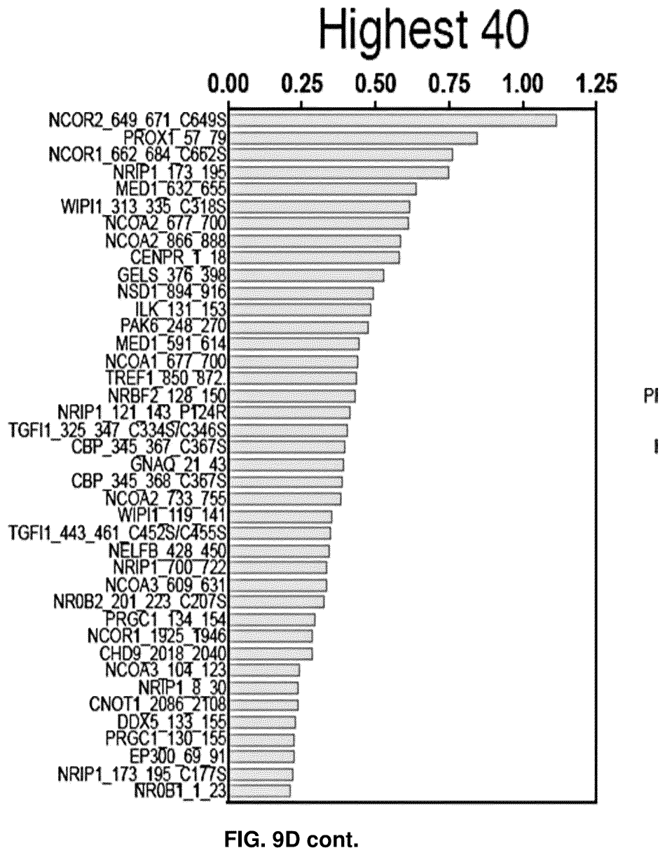

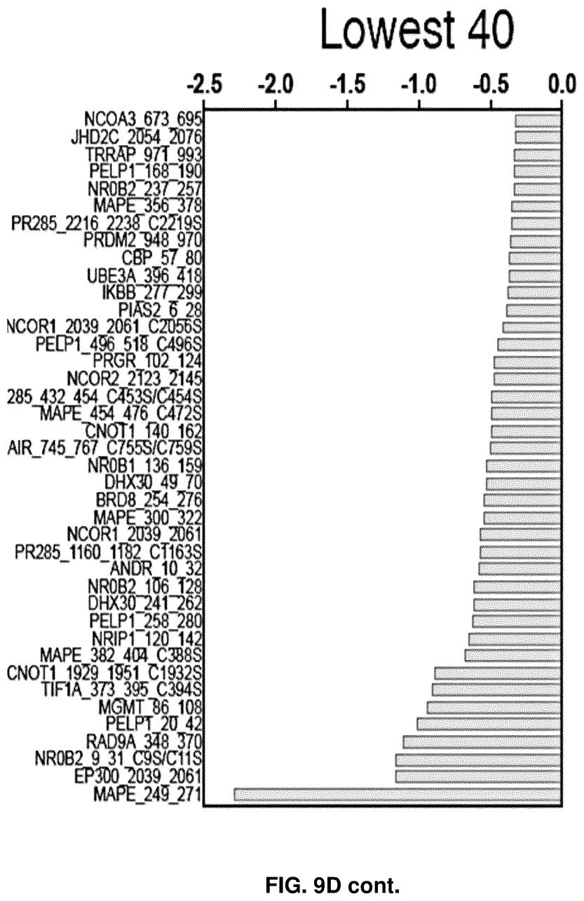

It was previously shown that mice with the human Gilbert's polymorphism are resistant to weight gain and hepatic steatosis. Using this model, WAT size and mitochondrial number were analyzed. In FIG. 9A, it is shown that the humanized Gilbert's polymorphism mice have lower WAT size and higher mitochondrial number. Similar to the results with PEG-BR, in FIG. 9B it is shown that the GS mice had no change on mitochondrial number. The GS mice do have higher PPARα expression in WAT and BAT (FIG. 9B). It was previously found that the liver of the GS mice also had higher PPARα expression because of reduced serine 73 phosphorylation of PPARα, which is known to cause ubiquitination and reduced expression. The serine 12 site of PPARα has been shown to be necessary for activation. In FIG. 9C, the GS mice have hyperphosphorylation of serine 12 of PPARα in WAT, and increased UCP1 and ADRB3 expression. The MARCONI nuclear hormone receptor analysis of endogenous PPARα in WAT of the GS and control mice revealed that PPARα has higher binding to coregulators and a unique molecular signature (FIG. 9D), which is similar to PEG-BR treated animals. The GS mice were comparable to the PEG-BR treated mice with higher binding with several coregulators, amino acids that are contained in nuclear receptor coactivators (NCOA2, NCOA3, NCOA1), nuclear receptor corepressors (NCOR1 and NCOR2), and peroxisome proliferator-activated receptor gamma coactivator 1-alpha (PGC-1a). NRIPI did appear for a lower interaction at −0.8 at amino acids 120-142, but not for the other sites that were observed with PEG-BR. In general, the coregulators with reduced binding in the GS mice were more diverse compared to the PEG-BR treated animals. Overall, the GS mice have hyperbilirubinemia that induces PPARα phosphorylation and a specific set of coregulators that mediate WAT size and mitochondrial number.

### Peg-BR Treatment Decreases Hepatic Lipid Accumulation

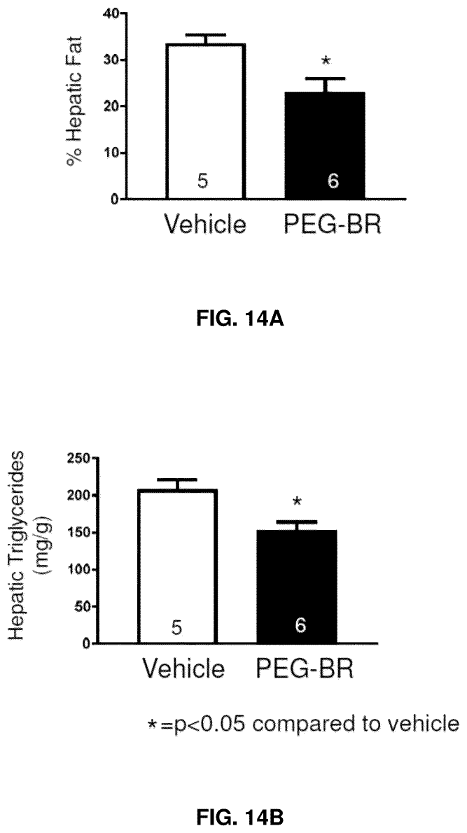

Studies were performed in male C57BL/6J mice that were fed 60% high fat diet (diet #D12492, Research Diets, Inc., New Brunswick, NJ) for 30 weeks. Mice were treated with PEG-BR (30 mg/kg, ip (n=6) or vehicle (saline, n=5) every other day for 4 weeks. At the end of the study, hepatic fat content was measured by EchoMRI and hepatic triglycerides were measured biochemically. As FIGS. 14A-14B show, PEG-BR treatment significantly decreased hepatic fat mass as detected by EchoMRI as compared to vehicle treated (33.5±1.5 vs. 23±3% vehicle vs. PEG-BR, p<0.05) and significantly increased lean mass as compared to saline treated (65 5±1 vs. 72 5±4%, vehicle vs. PEG-BR, p<0.05. PEG-BR also significantly decrease hepatic triglycerides as compared to vehicle treated mice (208±13, vs. 153±11 mg/g, p<0.05).

### DISCUSSION

Adipose depots differ in their functions but serve as integrators of metabolic and hormonal pathways that mediate energy balance and glucose homeostasis. For unknown reasons, bilirubin plasma levels are lower in the obese. How this affects adipose tissue stores is unknown. Bilirubin has been shown to be an antioxidant, but this function does not account for all the mechanistic lipid-lowering actions. These examples reveal that bilirubin functions as a metabolic hormone through a PPARα-dependent mechanism that improves WAT function. These examples show that mice with the human Gilbert's polymorphism and elevated bilirubin levels have paralleled reduced fat mass, and lower plasma insulin and glucose levels. It is shown herein that the GS mice have significantly higher PPARα expression and coregulator recruitment in WAT, including brown fat marker PGCla. PEG-BR increased mitochondrial function and number in WAT, which was found to also increase PPARα interaction with PGCla as well as nuclear receptor coactivators and corepressors. These interactions are important for gene regulator activity of PPARα.

Demonstrating these slight variances in gene regulation, the fibrates have been shown to be better at reducing inflammation than WY 14,643 and are typically used in treating inflammatory hyperlipidemia and fatty liver disease. While WY 14,643 does reduce hyperlipidemia, it does not reduce inflammation. However, WY 14,643 has been shown to be more efficient at lowering blood glucose levels. Bilirubin may likewise regulate a unique subset of PPARα target genes as a selective PPAR modulator (SPPARM) for PPARα that regulate its anti-obesity,-diabetic, and-cardiovascular properties in vivo.

### Animals

The experimental procedures and protocols of this example conform to the National Institutes of Health Guide for the Care and Use of Laboratory Animals, and were approved by the Institutional Animal Care and Use Committee of the University of Mississippi Medical Center in accordance with the NIH Guide for the Care and Use of Laboratory Animals. All mice had free access to food and water ad libitum. Animals were housed in a temperature-controlled environment with 12 h dark-light cycle. Diet-induced obese (DIO) mice were treated with the recently described water-soluble PEGylated BR (PEG-BR). PEG-BR treatment was performed on adult mice who were on 60% high-fat diet (diet #D12492, Research Diets, Inc., New Brunswick, NJ) for 36 weeks and allowed access to water. This diet contains 60% of its total kilocalories from fat and 20% from carbohydrates derived from mainly from maltodextrin 10 (12%) and sucrose (6.8%). Mice were then treated with PEG-BR (30 mg/kg, i.p., every other day) for 4 weeks. Gilbert's mice UGT1A1*28 (TgUGTA1*28) Ugt^{-/-} were as previously described.

### Body Composition

Body composition changes were assessed at 6-week intervals throughout the study using magnetic resonance imaging (EchoMRI-900TM, Echo Medical System, Houston, TX). MRI measurements were performed in conscious mice placed in a thin-walled plastic cylinder with a cylindrical plastic insert added to limit movement of the mice. Mice were briefly submitted to a low-intensity electromagnetic field where fat mass, lean mass, free water, and total water were measured.

### Fasting Glucose

Following an 8 hour fast, a blood sample was obtained via orbital sinus under isoflurane anesthesia. Blood glucose was measured using an Accu-Chek Advantage glucometer (Roche, Mannheim, Germany).

### Measurement of Total Bilirubin

Total bilirubin was measured from plasma using a Vet Axcel chemistry analyzer (Alfa Wassermann, Caldwell, NJ) according to manufactures guidelines. All reactions were performed in duplicate with standards supplied by the manufacturer and the data presented as mg/dL.

### Measurement of Triglycerides and Cholesterols

NMR experiments were acquired using a 14.0 T Bruker magnet equipped with a Bruker AV-III console operating at 600.13 MHz. All spectra were acquired in 3 mm NMR tubes using a Bruker 5 mm QCI cryogenically cooled NMR probe. Plasma samples were prepared and analyzed according to the Bruker In-Vitro Diagnostics research (IVDr) protocol. Sample preparation consisted of combining 50 μl of plasma with 150 μl of buffer supplied by Bruker Biospin specifically for the IVDr protocol. For 1D ¹H NMR, data was acquired using the 1D-NOE experiment which filters NMR signals associated with broad line widths such as those arising from proteins that might be present in plasma samples and adversely affect spectral quality. Experiment conditions included: sample temperature of 310 K, 96k data points, 30 ppm sweep width, a recycle delay of 4 s, a mixing time of 150 ms and 32 scans. Lipoprotein subclass analysis was performed using regression analysis of the NMR data which is done automatically as part of the IVDr platform.

### Mitotracker Mitochondrial Analysis

Frozen brown and white adipose tissue samples from the PEG-Bilirubin vs Control treated mice and the Humanized Gilbert's Syndrome vs. Control mice were thawed at room temperature. Specimen were then washed three times with prewarmed 37° C. PBS then incubated with 100 nM Mitotracker® Green FM (Invitrogen, location) for 15 min at room temperature. The samples were washed once with PBS, then incubated with 1 μM of Drag5 (Cell Signaling Technology, Danvers, Massachusetts), then washed one final time with PBS before imaging. The specimen images were taken using Confocal Microscopy.

### Measurement of Mitochondrial DNA Copy Number

Frozen brown and white adipose tissue samples from the PEG-Bilirubin vs. Control treated mice and the Humanized Gilbert's Syndrome vs. Control mice were thawed at room temperature. DNA was isolated using the GenElute™ Mammalian Genomic DNA Miniprep Kit Protocol (Millipore Sigma, location) according to manufacturer's instruction. The mtDNA Copy number was analyzed as relative mtDNA copy number via the ration of 16S rRNA, a mitochondrial gene, and GAPDH, a nuclear gene as previously described. PCR amplification of the genomic DNA was performed by quantitative real-time PCR using TrueAmp SYBR Green qPCR SuperMix (Advance Bioscience). The thermocycling protocol consisted of 3 min at 95° C., 48 cycles of 15 sec at 95° C., 30 sec at 60° C., and based on primer size 0 to 30 sec at 72° C.

### Lipid Droplet Sizes

Frozen brown and white adipose tissue samples from the PEG-Bilirubin vs. Control treated mice and the Humanized Gilbert's Syndrome vs. Control mice were thawed at room temperature. The images of the lipid droplet sizes were measured as previously described. Then tissue sample diameters were measured based on the measurement of the lipid droplet's widest point. The diameter was used to extrapolate the lipid volume for the adipocytes.

### Pamstation Nuclear Hormone (NHR) Assay

PPARα interactions with co-regulators was characterized with the PAMStation Nuclear Hormone Receptor Chip (PamChip no. 88011; Pamgene International). Each array was incubated with a reaction mixture of 5 nM GST-tagged PPARα-LBD (PV4692, Invitrogen), 25 nM Alexa488-conjugated anti-GST-antibody (Alexa488; Invitrogen; A11131), and TR-FRET Co-regulator buffer J (PV4692, A-11131, and PV4682; Invitrogen). In separate tubes each reaction mixture was supplemented with DMSO, 50 UM of WY 14,643, 50 μM fenofibrate, or 50 μM bilirubin. Incubation was performed at 37° C. for 5 minutes in 1.5 ml microtubes prior to placement on respective array for analysis in a PamStation96 (Pamgene International). PPARα binding was reflected via fluorescent signals recorded through the Pamstation96. The signals were transformed into tiff images and binding capacity was quantified using BioNavigator software (Pamgene International).

### Wat NHR Assay

For tissue analysis, frozen samples were retrieved and pooled based on treatment condition. Upon rupture via homogenization, with a phosphatase inhibitor and protease inhibitor in 200 μl of M-PER buffer and HEMG buffer (10 mM HEPES, 3 mM EDTA, 10 mM Sodium Molybdate, 10%, Glycerol), samples were spun down at 14,000 rpm for 5-10 min. Supernatants were prepared for protein concentration measurement in triplicate using the Pierce™ BCA Protein Kit (Thermo fisher Scientific, Wilmington, DE). Samples were measured at 512 nm using the SpectraMax Plus (Molecular Devices, San Jose, CA). A final amount of 25 ng of protein lysate, 12.5 nM anti-PPARα Antibody (Santa Cruz Biotechnology, catalog sc-1982), 77.5 nM anti-Goat 488 Alexa fluor (Fisher) were added to a microtube. To compare reactions, a pure ligand binding domain mixture was used on two of the arrays with a composition of 5 nM PPARα LBD, 50 μM of Bilirubin or Vehicle (DMSO), and 12.5 nM anti-PPARα Antibody (Santa Cruz Biotechnology, sc1982), and 77.5 nM anti-Goat 488 Alexa fluor (Fisher). Before placing on the array, all final mixtures rotated for 30 min at 4° C. PPARα binding was reflected via fluorescent signals recorded through the Pamstation96. The signals were transformed into tiff images and binding capacity was quantified using BioNavigator software (Pamgene International).

### Cell Lines and Culture

The mouse 3T3-L1, BAT, and Cos7 green kidney monkey cells were routinely cultured and maintained in Dulbecco's Modified Eagle's Medium (DMEM) containing 10% bovine calf serum (BCS) or fetal bovine serum (FBS) with 1% Antibiotic-Antimycotic (AA). The vector, PPARα, and PPARγ2 cell lines were developed as previously described.

### Crispr Mediated Knockout of PPARα in BAT Cells

CRISPR-Technology was employed in BAT as previously described to excise part of Exon 3 and Exon 4 of the PPARα gene to create a PPARα Knockout BAT cell line. Two sgRNAs with high efficacy and low off-target scores were identified on Exon 3 and 4 of the mouse PPARα gene using Benchling online software. The two Cas9 targets were separated by 9,465 bases. All of the off-targets to our PPARα sgRNA had 4 mismatches, of which at least 1-2 were within the seed region (up to 12 bases proximal to the protospacer adjacent motif (PAM) site) which reduces the likelihood of Cas9 off-target effects. The multiplex sgRNAs were generated using the PrecisionX Multiplex gRNA Cloning Kit according to manufacturer instructions. Oligonucleotides used are listed in Table 1. The multiplex sgRNA fragments were then cloned into the GuideIt Green plasmid according to the manufacturer's instructions. After sequence verification, 2 μg of the plasmid was transfected into cells in 12-well plates. After 36 h of transfection, cells with the top 10% level of fluorescence were single-sorted into 96-well plates by fluorescent activated cell sorting. After cells grew to confluence, individual wells were harvested with trypsin, and crude genomic DNA was obtained from two-thirds of the cells while the remaining one-third was left to continue growing. PCR was carried out on the genomic DNA samples using primers flanking the two cut sites (Exon 3,4; Table 1). Positive clones were identified by the presence of an 831-bp product (+/−depending on whether there is further insertion or deletion) indicative of Cas9-mediated targeting. Clones with the ˜316-bp product were sequentially expanded in 24-well and 6-well plates and then in 10-cm culture dishes.

TABLE 1
sgRNAa

| | | |
|---|---|---|
| PPARαsgRNAexn3-F | ccggGGAAGCTG | SEQ ID |
| | TCCGGGCTCCGA | NO: 1 |
| PPARαsgRNAexn3-R | aaacTCGGAGCC | SEQ ID |
| | CGGACAGCTTCC | NO: 2 |
| PPARαsgRNAexn4-F | ccggCATCGAGT | SEQ ID |
| | GTCGAATATGTG | NO: 3 |
| PPARαsgRNAexn4-R | aaacCACATATT | SEQ ID |
| | CGACACTCGATG | NO: 4 |

TABLE 2
Primers

| | | |
|---|---|---|
| PPARα-Exon3-Fwd | GCAGCTTGGC | SEQ ID |
| | ACCTTCTGTG | NO: 5 |
| PPARα-strdExn3-Rev | GATGACAGAG | SEQ ID |
| | CCCTCGGAGC | NO: 6 |
| PPARα-strdExn4-Fwd | GAGTGTCGAATA | SEQ ID |
| | TGTGGGGACAAG | NO: 7 |
| PPARα-Exon4-Rev | GCAACCTGCC | SEQ ID |
| | CTAGACTGTC | NO: 8 |

### Adipogenesis Assay

Adipogenic differentiation of 3T3-LI cells was achieved by treatment with 250 nM Dex, 167 nM insulin, and 500 μM isobutylmethylxanthine (IBMX) in 10% FBS until Day 9 as previously described. Adipogenic differentiation of BAT cells was achieved with 0.02 UM Insulin, 0.001 UM triiodothyronine (T3), 125u Indomethacin, 5.096 μM Dexamethasone, and 0.5 mM IBMX in 10% FBS until Day 10. Upon differentiation, cells were stained with Nile Red to visualize lipid content, and densitometry was used as a direct measure as previously described. Total RNA was extracted from Nile Red stained cells and used for real time PCR analysis.

### Quantitative Real-Time PCR Analysis

Total RNA was extracted from mouse tissues using the miRNeasy Mini Kit (Qiagen). Total RNA was read on a NanoDrop 2000 spectrophotometer (Thermo Fisher Scientific, Wilmington, DE) and cDNA was synthesized using High Capacity cDNA Reverse Transcription Kit (Applied Biosystems). PCR amplification of the cDNA was performed by quantitative real-time PCR using TrueAmp SYBR Green qPCR SuperMix (Advance Bioscience). The thermocycling protocol consisted of 3 min at 95° C., 48 cycles of 15 sec at 95° C., 30 sec at 60° C., and based on primer size 0 to 30 sec at 72° C. and finished with a melting curve ranging from 60-95° C. to allow distinction of specific products. Normalization was performed in separate reactions with primers to 36B4.

TABLE 3

| Gene | Genebank | | | | |
|---|---|---|---|---|---|
| Name | Number | Forward | | Reverse | |
| Ucp1 | NM_009463.3 | CAGCTTTGCC | SEQ ID | GAGGCAGGTG | SEQ ID |
| | | TCACTCAGGA | NO: 9 | TTTCTCTCCC | NO: 10 |
| Cpt1a | | GGCCTCTGTGG | SEQ ID | CTCAGTGGGAG | SEQ ID |
| | | TACACGACAA | NO: 11 | CGACTCTTCA | NO: 12 |
| FABP4 | NM_024406.3 | AGCTGGTGGT | SEQ ID | TTCCTTTGGC | SEQ ID |
| | | GGAATGTGTT | NO: 13 | TCATGCCCTT | NO: 14 |
| Cd36 | NM_001159558.1 | TCTTGGCTACAG | SEQ ID | AGCTATGCAGCA | SEQ ID |
| | | CAAGGCCAGATA | NO: 15 | TGGAACATGACG | NO: 16 |
| FGF21 | | | | | |
| Angptl4 | NM_020581.2 | GACGCCTGAA | SEQ ID | TCTCCGAAGCC | SEQ ID |
| | | CGGCTCTGT | NO: 17 | ATCCTTGTAG | NO: 18 |
| Adrb3 | NM_013462.3 | CCTTCCGTCG | SEQ ID | CCATCAAACC | SEQ ID |
| | | TCTTCTGTGT | NO: 19 | TGTTGAGCGG | NO: 20 |
| PPARα | NM_011144 | GGTGTTCGCA | SEQ ID | GGTGAGATAC | SEQ ID |
| | | GCTGTTTTGG | NO: 21 | GCCCAAATGC | NO: 22 |
| 36B4 | NM_007475.5 | CACTCTCGCT | SEQ ID | ACGCGCTTGT | SEQ ID |
| | | TTCTGGAGGG | NO: 23 | ACCCATTGAT | NO: 24 |

### Chromatin Immunoprecipitation (ChIP)

Differentiated BAT or 3T3-L1 cells were treated for 2 to 24 hours with DMSO, 50 μL WY-14,643, 50 μL Fenofibrate, or 50 μL Biliverdin. Cells were crosslinked with formaldehyde with a final concentration of 1% in media while shaking at room temperature for 10 min. The activity of the formaldehyde was quench with the addition of glycine while rocking for 5 min at room temperature. Cells were washed twice with 1×PBS, collected into a 15 ml conical tube and spun down at 3,000 rpm for 5 min. Pellets were rapidly frozen on dry ice ethanol mix and stored at −80° C. for a minimum of 1 hour or immediately resuspended in a series of lysis buffers (see table for ChIP buffer table) containing protease inhibitors for 5 min. Cells were sonicated for approximately 8 min per sample. The lysates were centrifuged for 10 min at 4° C. at 13,000 rpm. The lysates were pre-cleared in BSA/Salmon sperm blocked beads rotating for 2 hours at 4° C. After pre-clearing the lysate was transferred to another tube containing the PPARα (Abcam ab191226), IgG (Calbiochem NI01-100 μg), or GFP (Santa Cruz sc-9996) antibody and were rotated overnight at 4° C. Lysates were then incubated with Agarose A beads and rotated for 4 hours at 4° C. The samples were then washed with a ChIP washing buffer (see table for ChIP buffer table) 5 times. The protein was eluted in an Elution buffer at 65° C. for 30 min shaking every 2 min. The eluted samples were transferred to another tube and incubated at 65° C. overnight to reverse crosslinking. The samples were purified after 1-hour incubation with Proteinase K at 55° C. with a isopropanol/chloroform/ethanol mixture. DNA was quantified on a NanoDrop 2000 spectrophotometer (Thermo Fisher Scientific, Wilmington, DE). PCR amplification of the genomic DNA was performed by quantitative real-time PCR using TrueAmp SYBR Green qPCR SuperMix (Advance Bioscience). The thermocycling protocol consisted of 2 min at 50° C. and then 10 min at 95° C., 48 cycles of 30 sec at 95° C., 1 min at 65° C.

TABLE 4
ChIP buffer table

| Name | Recipe |
|---|---|
| Lysis Buffer 1 (LB1) | 50 mM HEPES-KOH, pH 7.5, 140 mM NaCl, |
| | 1 mM EDTA, pH 8.0, 10% glycerol, 0.5% |
| | NP-40, 0.25% Triton X-100 |
| Lysis Buffer 2 (LB2) | 10 mM Tris-HCl, pH 8.0, 200 mM NaCl, |
| | 1 mM EDTA, pH 8.0, 0.5 mM EGTA |
| Lysis Buffer 3 (LB3) | 10 mM Tris-HCl, pH 8.0, 100 mM NaCl, |
| | 1 mM EDTA, pH 8.0, 0.5 mM EGTA, |
| | 0.1% Na-Deoxycholate, 0.5% N-lauroylsarcosine |
| Wash Buffer (RIPA) | 50 mM HEPES-KOH, pka 7.55, 500 mM LiCl, |
| | 1 mM EDTA, pH 8.0, 1.0% NP-40, |
| | 0.7% Na-Deoxycholate |

TABLE 5
ChIP primer sequences

| Target | Forward | | Reverse | |
|---|---|---|---|---|
| Name | Sequence | | Sequence | |
| Adrb3 | GATCTCATGG | SEQ ID | TTGTGCTGAT | SEQ ID |
| (-4697/ | AGCCCAGACT | NO: 25 | TCATGCCTGT | NO: 26 |
| -4607) | | | | |
| Cpt1 | TTCACTGGGT | SEQ ID | TGGCATTGTCG | SEQ ID |
| (-3306/ | GCTCGGGAAG | NO: 27 | CAAGGATAAC | NO: 28 |
| -3109) | | | | |
| Ucp1 | GCAACCCTCT | SEQ ID | GCCTAACACC | SEQ ID |
| (-13K) | CCCATCAGTG | NO: 29 | GTGCTTCTCA | NO: 30 |

### Seahorse Cellular Respiration Analysis

Cellular respiration was quantified using the Seahorse Extracellular Flux Analyzer XF-96 (Agilent Technologies, Cedar Creek, TX). The Seahorse XF Cell Mito Stress Test Kit (Agilent Technologies, Cat #103015-100) was used for analysis of cellular respiration. BAT or 3T3-L1 cells were seeded on a XF96 cell culture microplate (Agilent 101085-004) at 20,000 cells per well. Cells were then differentiated as previously described for 9 days. Differentiated BAT or 3T3-LI cells were treated for 24 hours with DMSO, 50 μL WY-14,643, 50 μL Fenofibrate, or 50 μL Biliverdin before analysis via Seahorse Instrument. The oxygen consumption rate (OCR) and extracellular acidification rate (ECAR) were used to quantify the cellular energy phenotype of the cells. After treatment, cells were washed twice with Seahorse Bioscience Assay Media (XF Base media with 25 mM Glucose, 2 mM L-Glutamate, and 1 mM Sodium Pyruvate) then incubated with the buffer for 1 hour in a non-CO₂ incubator. The Seahorse Cartridge ports were loaded with 20 mL of assay media with 10 PM FCCP, 10 μM Oligomycin, 5 μM Rotenone/Antimycin A in different ports an hour before assay. Treatment performed via the devices followed by sequential measurements, resulted in obtaining the baseline respiration, ATP production, Maximal respiration, Proton Leak, and Non-Mitochondrial respiration. The raw data and graphs were supplied as an Excel File or Graphpad Prizm file.

### Promoter Reporter Assays

An expression vector for Flag-Tagged PPARα was constructed as previously described. The cells were transfected with RXR-SG5 and either WT-Flag PPARα or with a Flag-Tagged PPARα plasmid with one of the following mutations: M330G, A333G, or T283G, in order to determine if binding of bilirubin at previously predicted positions would alter activity. Cells were also transfected with RXR-SG5 to enhance PPAR activity and the PPAR minimal reporter promoter plasmid (3Tk-Luc), whose activity was measured by luciferase, and pRL-CMV Renilla reporter for normalization to transfection efficiency. Transient transfection was achieved using GeneFect (Alkali Scientific, Inc.) during a 24-hour span. Cells were then treated for 24 hours with DMSO, 50 μL WY-14,643, 50 μL Fenofibrate, or 50 μL Biliverdin, then cells were lysed, and the luciferase assay was performed using the Promega dual luciferase assay system (Promega, Madison, WI).

### Whole Cell Extraction

Cells were washed and collected in 1×PBS followed by centrifugation at 1500×g for 5 min. The supernatant was discarded and the pellet was re-suspended in 1×PBS. After a short spin at 13,000 rpm for 2 min at 4° C. the pellet was rapidly frozen on dry ice ethanol mix and stored at −80° C. for a minimum of 1 hour. The frozen pellet was then re-suspended in 3 volumes of cold whole cell extract buffer (20 mM HEPES, 25% glycerol, 0.42M NaCl, 0.2 mM EDTA, pH 7.4) with protease inhibitors and incubated on ice for 30 min. The samples were centrifuged at 45,000 rpm for 7 min at 4° C. Supernatants were prepared for protein concentration measurement in triplicate using the Pierce™ BCA Protein Kit (Thermo fisher Scientific, Wilmington, DE). Samples were measured at 512 nm using the SpectraMax Plus (Molecular Devices, San Jose, CA). The supernatants were either stored at −80° C. or used immediately for Western analysis to determine protein expression levels.

### Gel Electrophoresis and Western Blotting

Supernatants from WCE were resolved by SDS polyacrylamide gel electrophoresis and electrophoretically transferred to Immobilon-FL membranes. Membranes were blocked at room temperature for 1 hour in Odyssey Blocking buffer (LI-COR Biosciences, Lincoln, NE) or TBS [TBS; 10 mM Tris-HCl (pH 7.4) and 150 mM NaCl] containing 5% BSA or milk. Subsequently, the membrane was incubated overnight at 4° C. with PPARα (Santa Cruz Biotechnology, Santa Cruz, CA, sc-9000), PPARγ (Santa Cruz Biotechnology, Santa Cruz, CA, sc-7273), PPARγ2 (Santa Cruz Biotechnology, Santa Cruz, CA, sc-22020), or HSP90 antibodies (Santa Cruz Biotechnology, Santa Cruz, CA, sc-13119). After three washes in TBST (TBS plus 0.1% Tween 20), the membrane was incubated with an infrared anti-rabbit (IRDye 800, green) or anti-mouse (IRDye 680, red) secondary antibody labeled with IRDye infrared dye (LI-COR Biosciences) (1:15,000 dilution in TBS) for 2 hours at 4° C. Following an additional 3 washes in TBST, immunoreactivity was visualized and quantified by infrared scanning in the Odyssey system (LI-COR Biosciences, Lincoln, NE).

### Statistical Analysis

Data were analyzed with Prism 7 (GraphPad Software, San Diego, CA) using analysis of variance combined with Tukey's post-test to compare pairs of group means or unpaired t tests. Results are expressed as mean±SEM. Additionally, one-way ANOVA with a least significant difference post hoc test was used to compare mean values between multiple groups, and a two-tailed, and a two-way ANOVA was utilized in multiple comparisons, followed by the Bonferroni post hoc analysis to identify interactions. p values of 0.05 or smaller were considered statistically significant.

### Preparation of PEG-BR Conjugate

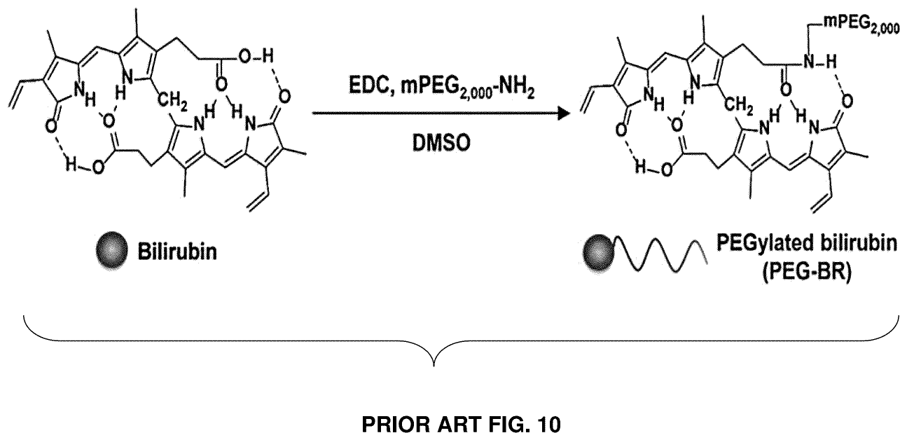

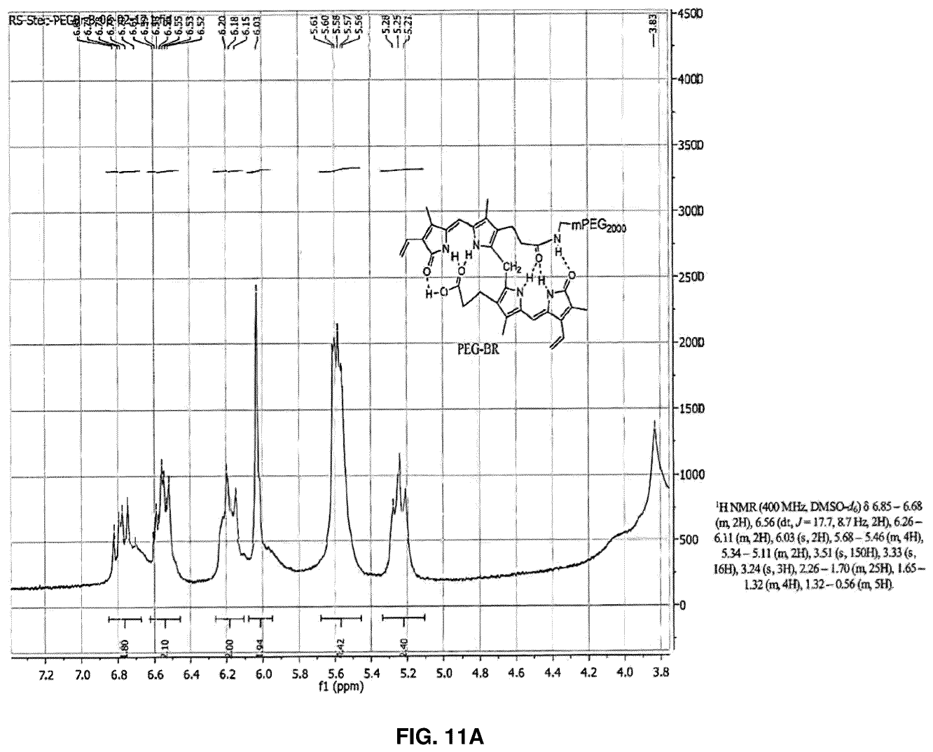

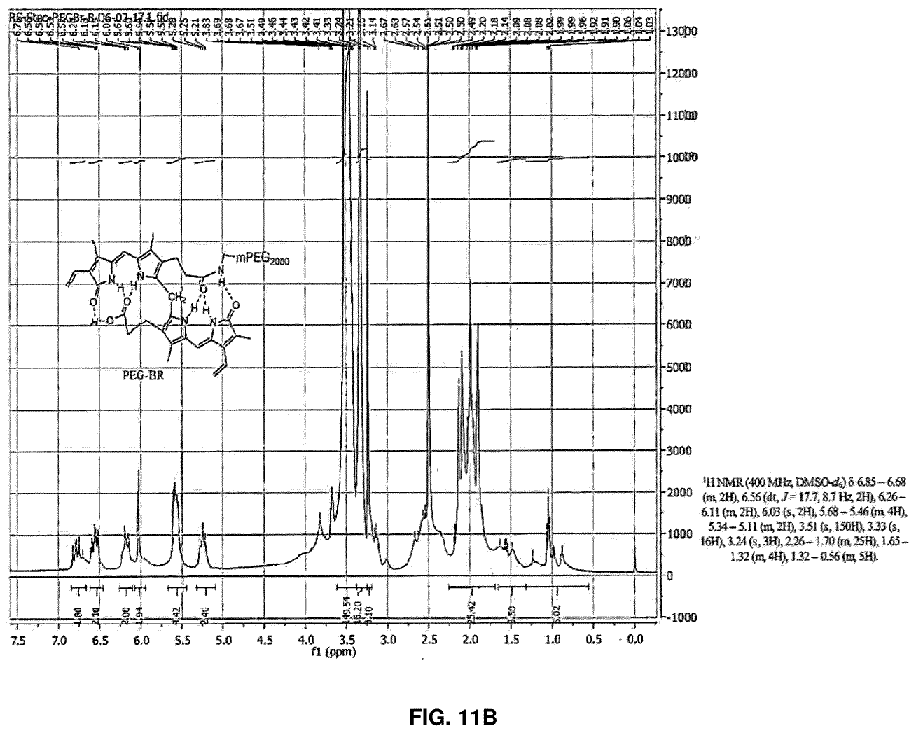

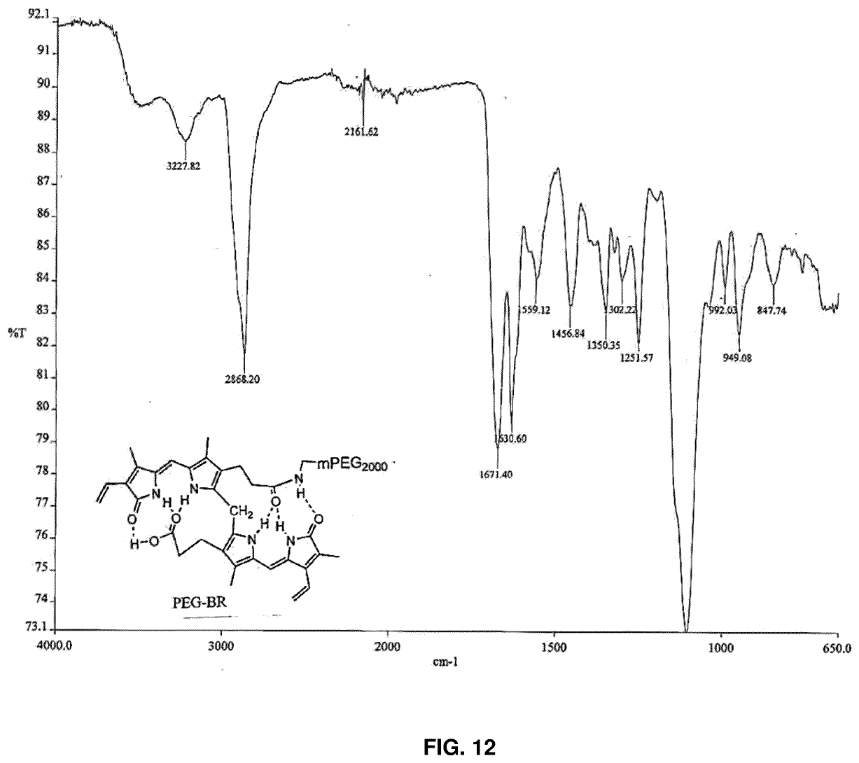

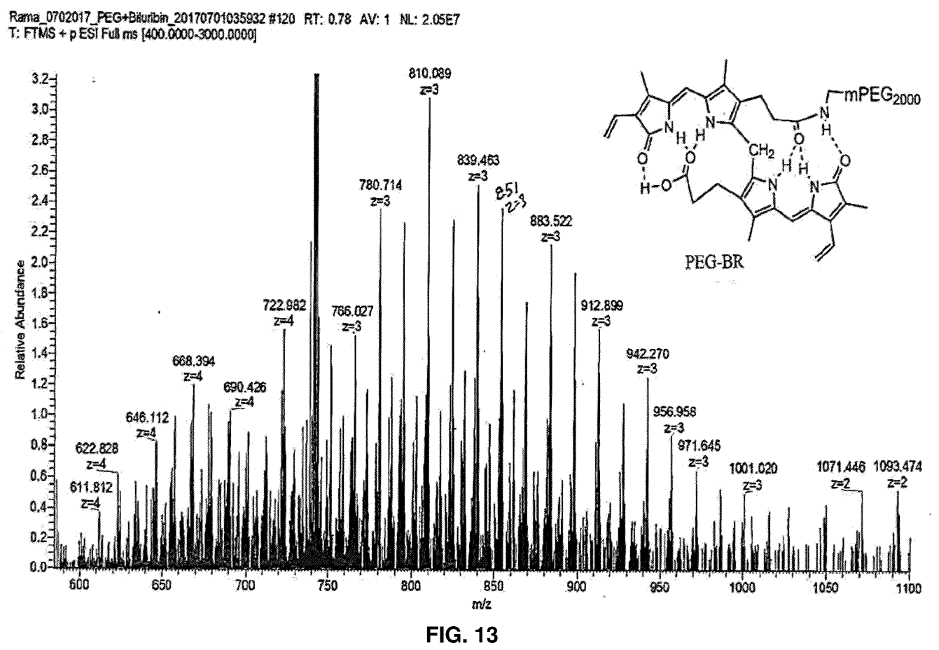

Bilirubin (alpha) (2.34 g; 4 mmol; Frontier Scientific) and 1-ethyl-3-(3-dimethylaminopropyl carbodiimide (EDC; 0.921 g; 4.8 mmol; Sigma-Aldrich Co.) were dissolved in dimethyl sulfoxide and stirred for 10 minutes at room temperature. Then, methoxy PEG 2000-amine (mPEG2000-NH₂; 3.3 g; 1.6 mmol; Layson Bio Inc.) and trimethylamine (1.2 ml) were added and stirred for 4 hours at room temperature under an argon atmosphere. Then, to the reaction mixture chloroform (1.5 L) was added and washed with 0.1 M HCl, 0.1 M NaOH, and 5% NaHCO₃ sequentially using a separatory funnel. The organic layer was dried using anhydrous sodium sulfate, filtered, and concentrated under rotavap to get 2.791 g of PEGylated bilirubin (PEG-BR). Purity of the conjugate was confirmed by proton NMR (using DMSO-d₆ as the solvent), IR and Mass spectral analysis. The ¹H NMR spectra are shown in FIGS. 11A-11B. The IR spectrum is shown in FIG. 12. The mass spectrum is shown in FIG. 13.

Mass peaks are charged 3 with positive ion peak m/z 851 and the mass was observed to be (C₁₂₃H₂₁₇N₅O₄₉) 2553 (calculated mass: 2548). ¹H NMR (400 MHZ, DMSO-d₆) δ 6.85-6.68 (m, 2H), 6.56 (dt, J=17.7, 8.7 Hz, 2H), 6.26-6.11 (m, 2H), 6.03 (s, 2H), 5.68-5.46 (m, 4H), 5.34-5.11 (m, 2H), 3.51 (s, 150H), 3.33 (s, 16H), 3.24 (s, 3H), 2.26-1.70 (m, 25H), 1.65-1.32 (m, 4H), 1.32-0.56 (m, 5H).

The produced PEG-BR has the following structural formula:

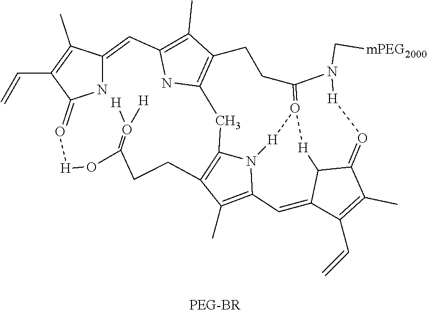

### Procedure to Make BRNPs (Nanoparticles)

A nice film layer of PEG-BR (accurately about 200 mg) was made in each vial with a vial capacity of 32 ml using chloroform and dried under a stream of argon and further dried under vacuum pump for 6 hours. Then, PBS buffer (1 ml) was added for every 10 mg of PEG-BR conjugate. For instance, a vial with 200 mg of PEG-BR was added with 20 ml, and a vial with 133 mg was added with 13.3 ml, of the buffer. The resulting suspension was sonicated for about ten minutes to yield uniformly sized BRNPs.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition. many modifications may be made to adapt a particular situation or material to the teachings of the disclosure without departing from the essential scope thereof.

What is claimed is:

1. A method for decreasing one or more of body weight, total fat, percent fat mass, visceral fat, epididymal fat, hepatic fat content, fasting blood glucose, low density lipoprotein (LDL) cholesterol, or plasma triglyceride levels, the method comprising:
administering an effective amount of PEGylated bilirubin to a subject, and
decreasing one or more of body weight, total fat, percent fat mass, visceral fat, epididymal fat, hepatic fat content, fasting blood glucose, LDL cholesterol, and plasma triglyceride levels in the subject.

2. The method of claim 1, wherein the subject is a human.

3. The method of claim 1, wherein the PEGylated bilirubin comprises bilirubin nanoparticles.

4. The method of claim 1, wherein the PEGylated bilirubin comprises the following structure

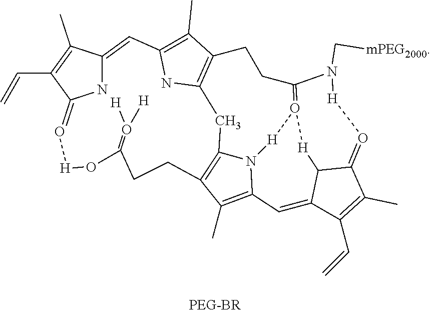

5. A method for decreasing white adipose fat (WAT) adipocyte size, the method comprising administering an effective amount of PEGylated bilirubin to a subject, and decreasing WAT adipocyte size of the WAT cells in the subject.

6. The method of claim 5, wherein the subject is a human.

7. The method of claim 5, wherein the PEGylated bilirubin comprises bilirubin nanoparticles.

8. The method of claim 5, wherein the PEGylated bilirubin comprises the following structure

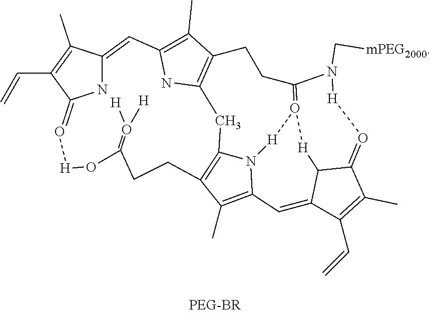

9. A method for increasing expression of UCP1 or ADRB3 in white adipose fat (WAT), the method comprising administering an effective amount of PEGylated bilirubin to WAT cells, and increasing expression of UCP1 or ADRB3 in the WAT cells.

10. The method of claim 9, wherein the PEGylated bilirubin comprises bilirubin nanoparticles.

11. The method of claim 9, wherein the subject is a human.

12. The method of claim 9, wherein the PEGylated bilirubin comprises the following structure

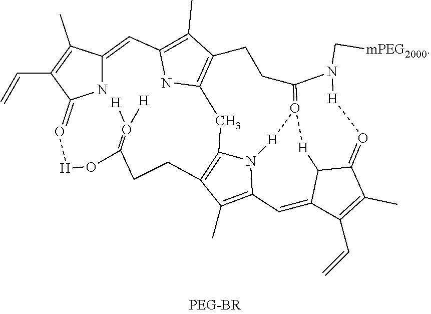

13. A method for increasing mitochondrial function and number in white adipose fat (WAT) cells, the method comprising administering an effective amount of PEGylated bilirubin to WAT cells and increasing mitochondrial function and number in the WAT cells.

14. The method of claim 13, wherein the PEGylated bilirubin comprises bilirubin nanoparticles.

15. The method of claim 13, wherein the PEGylated bilirubin comprises the following structure [chemical structure]
